(19) United States
(12) Patent Application Publication
(10) Pub. No.: US 2020/0399390 A1
(43) Pub. Date: Dec. 24, 2020

(54) METHODS FOR PRODUCING LYMPHOCYTE PROGENITORS

(71) Applicant: MURDOCH CHILDRENS RESEARCH INSTITUTE, Parkville, Victoria (AU)

(72) Inventors: Edouard Guy STANLEY, Ascot Vale (AU); Andrew ELEFANTY, Surrey Hills (AU); Ali MOTAZEDIAN, Parkville (AU)

(73) Assignee: MURDOCH CHILDRENS RESEARCH INSTITUTE, Parkville, Victoria (AU)

(21) Appl. No.: 16/492,036

(22) PCT Filed: Mar. 8, 2018
(86) PCT No.: PCT/AU2018/050210

§ 371 Date: Sep. 6, 2019

(30) Foreign Application Priority Data
Mar. 8, 2017 (AU) 2017900805

(51) Int. Cl. C07K 16/28; C07K 16/22; C12N 5/0781; C07K 14/54

(52) U.S. Cl. CPC C07K 16/2896; C07K 16/2809; C07K 16/22; C12N 2506/45; C07K 14/5418; C12N 2506/02; C12N 5/0635

(57) ABSTRACT

The invention relates to a method for producing a lymphocyte progenitor, the method comprising culturing a pluripotent stem cell (PSC)-derived CD34+ cell at an air-liquid interface (ALI). The invention also relates to a method for producing a B-cell progenitor, the method comprising co-culturing a PSC-derived CD34+ cell and a stromal cell in a medium comprising a CD117 activator and a NOTCH1 inhibitor. The invention further relates to a T-cell progenitor and a B-cell progenitor when produced by the methods of the invention and to use of the T-cell progenitor in the manufacture of T cell with defined antigen specificity, optionally a chimeric antigen receptor (CAR) T cell, and use of the B-cell progenitor in the manufacture of an antibody.

### CROSS-REFERENCE TO RELATED APPLICATIONS

This Application is a 35 U.S.C. § 371 National Phase Entry Application of International Application No. PCT/AU2018/050210 filed on Mar. 8, 2018, which designated the U.S., and which claims benefit of Australian Application No. 2017900805 filed on Mar. 8, 2017, the contents of which are incorporated herein by reference in their entireties.

### FIELD

The invention relates to methods for producing a lymphocyte progenitor, optionally a T-cell progenitor or a B-cell progenitor, a T-cell progenitor or a B-cell progenitor produced by the methods, and use of the T-cell progenitor or the B-cell progenitor in the manufacture of therapeutic agents.

### BACKGROUND

Human pluripotent stem cells (PSCs), including embryonic stem cells (ESCs) and induced pluripotent stem cells (iPSCs), are cell types that can undergo unlimited self-renewal and, in theory, have the ability to give rise to all of the different cell types in the body. These properties of PSCs make them an attractive platform for generating a variety of normal and mutated human cell types for medical related applications.

Cells of B-cell and T-cell lineages are also important for medical therapy.

Accordingly, there is a need for improved production of cells of B-cell and T-cell lineages from PSCs.

It is to be understood that if any prior art publication is referred to herein, such reference does not constitute an admission that the publication forms a part of the common general knowledge in the art in Australia or any other country.

### SUMMARY

A first aspect provides method for producing a lymphocyte progenitor, the method comprising culturing a pluripotent stem cell (PSC)-derived CD34⁺ cell at an air-liquid interface (ALI).

In one embodiment of the method of the first aspect, culturing the CD34⁺ cell excludes co-culturing the CD34⁺ cell with an exogenous stromal cell, optionally a non-syngeneic mouse or human stromal cell.

In one embodiment of the method of the first aspect, the method excludes purifying the CD34⁺ cell before culturing. In one embodiment, the method comprises generating an embryoid body. In one embodiment, the embryoid body is generated by culturing the PSCs cell in a medium comprising a WNT agonist, optionally CHIR99021. In one embodiment, the embryoid body is generated by culturing the PSCs cell in a medium comprising a WNT agonist, optionally CHIR99021, from day about zero to about day 2, or from about day 2 to about day 4, or from about day zero to about day 4.

In one embodiment, the method of the first aspect comprises culturing at the ALI for about 2 weeks to about 5 weeks.

In one embodiment of the method of the first aspect, the PSC is human, optionally an embryonic stem cell (ESC) or induced PSC (iPSC).

In one embodiment of the method of the first aspect, the lymphocyte progenitor is a T-cell progenitor, and the method comprises culturing the CD34⁺ cell at the ALI in a medium comprising vascular endothelial growth factor (VEGF), basic fibroblast growth factor (bFGF, FGF2), stem cell factor (SCF), fms-related tyrosine kinase 3 ligand (FLT3L), and interleukin-7 (IL-7), and optionally IL-3.

In one embodiment of the method of the first aspect, the lymphocyte progenitor is a T-cell progenitor, and the medium excludes IL-3 or IL-6.

In one embodiment of the first aspect, the lymphocyte progenitor is a B-cell progenitor, and the method comprises culturing the CD34⁺ cell in a medium comprising a NOTCH1 inhibitor during or after an embryoid body comprising the CD34⁺ cell is cultured at the ALI. In one embodiment, the method comprises culturing the embryoid body comprising the CD34⁺ cell in a medium comprising the NOTCH1 inhibitor from about day 15, optionally to about day 32, during culturing at the ALI. In one embodiment, the method comprises culturing the embryoid body comprising the CD34⁺ cell at the ALI in a medium comprising a CD117 activator.

In one embodiment of the first aspect, the lymphocyte progenitor is a B-cell progenitor, and the method comprises culturing the embryoid body comprising the CD34⁺ cell in the medium comprising a CD117 activator.

In one embodiment of the first aspect, the lymphocyte progenitor is a B-cell progenitor, and the method further comprises culturing in a medium comprising IL-7 from about day 8. In one embodiment, the method comprises culturing in a medium comprising IL-7 for up to 35 days, optionally 30 days.

In one embodiment of the first aspect, the NOTCH1 inhibitor is N—[N-(3,5-difluorophenacetyl)-L-alanyl]-S-phenylglycine t-butyl ester (DAPT).

In one embodiment of the first aspect, the CD117 activator is stem cell factor (SCF).

A second aspect provides a lymphocyte progenitor when produced by the method of the first aspect.

A third aspect provides use of a lymphocyte progenitor when produced by the method of the first aspect in the manufacture of (i) a T cell with defined antigen specificity, optionally a chimeric antigen receptor (CAR) T cell or (ii) an antibody.

A fourth aspect provides a method for producing a B-cell progenitor, the method comprising co-culturing a pluripotent stem cell (PSC)-derived CD34⁺ cell and a stromal cell in a medium comprising a CD117 activator and a NOTCH1 inhibitor. In one embodiment, the method further comprises co-culturing the cells in a medium comprising interleukin-7 (IL-7).

In one embodiment of the fourth aspect, the method comprises co-culturing the cells in the medium comprising a CD117 activator and a NOTCH1 inhibitor from about day 15. In one embodiment, the method comprises co-culturing the cells in the medium comprising a CD117 activator and a NOTCH1 inhibitor from about day 15 for about 14 to about 28 days, optionally 21 days.

In one embodiment of the fourth aspect, the method comprises co-culturing in a medium comprising IL-7 from about day 15. In one embodiment, the method comprises (ii) co-culturing in a medium comprising IL-7 for about 14 days to about 28 days, optionally 21 days.

In one embodiment of the method of the fourth aspect, the cells are co-cultured in a medium comprising IL-7, a CD117 activator and a NOTCH1 inhibitor.

In one embodiment of the method of the fourth aspect, the cells are co-cultured in a medium comprising IL-7 and a CD117 activator, but excluding a NOTCH1 inhibitor.

In one embodiment of the method of the fourth aspect, the cells are (a) co-cultured in a medium comprising IL-7, a CD117 activator and a NOTCH1 inhibitor and then (b) co-cultured in a medium comprising IL-7 and a CD117 activator, but excluding a NOTCH1 inhibitor.

In one embodiment of the fourth aspect, the method excludes purifying the CD34⁺ cell before co-culturing.

In one embodiment of the fourth aspect, the method comprises co-culturing an embryoid body and the stromal cell.

In one embodiment of the method of the fourth aspect, the CD117 activator is stem cell factor (SCF).

In one embodiment of the fourth aspect, the NOTCH1 inhibitor is N—[N-(3,5-difluorophenacetyl)-L-alanyl]-S-phenylglycine t-butyl ester (DAPT).

In one embodiment of the method of the fourth aspect, the stromal cell expresses delta like canonical notch ligand 4 (DLL4).

In one embodiment of the method of the fourth aspect, the stromal cell is an OP9, MS5 or S17 cell. In one embodiment, the stromal cell is an OP9-DLL4 cell.

In one embodiment of the method of the fourth aspect, the PSC is human, optionally an embryonic stem cell (ESC) or induced PSC (iPSC).

A fifth aspect provides a B-cell progenitor when produced by the method of the fourth aspect.

A sixth aspect provides use of a B-cell progenitor when produced by the method of the fourth aspect in the manufacture of an antibody.

### BRIEF DESCRIPTION OF THE FIGURES

FIG. 1. Overview of current strategies for lymphocyte differentiation from PSCs to B-lymphocytes includes the stepwise differentiation of PSCs toward a CD34⁺ hemogenic endothelium population followed by culture on stromal layers in presence of suitable cytokines. As shown here, two fundamentally different protocols have been described for generating CD34⁺ hematopoietic progenitors from PSCs: co-culturing PSCs on mouse stromal cells (e.g. OP9, MS5, S17) or embryoid body (EB) formation. While the current protocols readily support NK-cell generation and T-lineage induction, reports of B-lineage induction are few (n=4), which reflects the complexity of hematopoiesis.

FIG. 2. Overview of PSC differentiation models proceeding via mesoderm and haematopoietic progenitor cells (HPC).

FIG. 3. Overview of B-lymphopoiesis from a CD34⁺ multi-lymphoid progenitor (MLP) cell.

FIG. 4. Photomicrographs showing generation of aorta-gonad-mesonephros (AGM) from PSCs.

FIG. 5. Overview of T-lymphopoiesis from a CD34⁺ multi-lymphoid progenitor (MLP) cell according to the disclosure. Blood lineages are derived from hemato-endothelial progenitors (hemogenic endothelium). Hemogenic endothelium expresses DLL4. Hematopoietic cells down-regulate DLL4 expression. Endothelial cells maintain DLL4 expression. Blood cells may be exposed to DLL4 expressed on proximal endothelial cells, potentially meeting conditions necessary for T-cell commitment. It is possible to detect early T-cell progenitors in cultures that generate definitive blood progenitors.

FIG. 6. Overview of production of RAG1-GFP PSC lines.

FIG. 7. Overview of T-lymphopoiesis from PSCs.

FIG. 8. Photomicrograph and flow cytometry plots showing T-lymphopoiesis from PSCs in liquid culture.

FIG. 9. Schematic representation of T-lymphopoiesis from PSCs using air-liquid interface (ALI) culture.

FIG. 10. Schematic representation and flow cytometry plots of T-lymphopoiesis from PSCs using ALI culture showing that ALI is a superior platform that enabled the generation of T-cells.

FIG. 11. Flow cytometry plots of T-lymphopoiesis from PSCs using ALI culture.

FIG. 12. Flow cytometry plots of T-lymphopoiesis from induced PSCs (iPSCs) using ALI culture.

FIG. 13. Summary of T-lymphopoiesis from PSCs according to the present disclosure.

FIG. 14. Schematic representation and flow cytometry plots showing generation of CD3+ immature T-cells.

FIG. 15. Schematic representation B-lymphopoiesis via blocking T-lymphopoiesis.

FIG. 16. Schematic representation and flow cytometry plots showing one example of B-lymphopoiesis according to the disclosure.

FIG. 17. Schematic representation and flow cytometry plots of a preferred embodiment of improved B-lymphopoiesis according to the invention.

FIG. 18. Flow cytometry plots showing compatibility of B-lymphopoiesis methods of the disclosure.

FIG. 19. Flow cytometry analysis of human thymocytes from an immunocompromised mouse injected with either RAG1⁺ iPSC derived human T-cell progenitors or unfractionated human cord blood. Panels show analysis for expression of CD4, CD8 and CD3. Note that surface expression of CD3 is indicative of completed TCR rearrangement. Gates set relative to CD45⁻ populations.

FIG. 20. Clonal expansion of B-cell progenitors on OP9 stromal cells (A) and appearance of a small fraction of HLADR⁺IgM⁺RAG1⁺ cells at day 42 (B). RAG1⁺CD19⁺CD10⁺ cells arising in stromal free ALI cultures (C).

### DETAILED DESCRIPTION

In this study, methods for generating B-cells and T-cells from PSCs were investigated. B and T lymphocytes together comprise the adaptive immune system. One of the functions of T-cells is to find and destroy defective cells, a function that has been co-opted into promising cell based therapies for the treatment of CD19⁺ leukaemia. Similarly, antibodies produced by B-cells now underlie promising treatments for a range of malignancies, including melanoma and lung cancer. Moreover, B-lymphocytes are of particular interest as their derivation would be indicative of progress towards the development of conditions that support hematopoietic stem cell (HSC) formation, the cell type which is likely to have the most immediate clinical applications. Therefore, in vitro production of both T- and B-cells from PSCs will facilitate development of a new generation of therapeutics derived from these cell types. In this disclosure, growth factor mediated induction of B-lymphoid cells as well as culture conditions that supported “stromal free” T-cell development were examined.

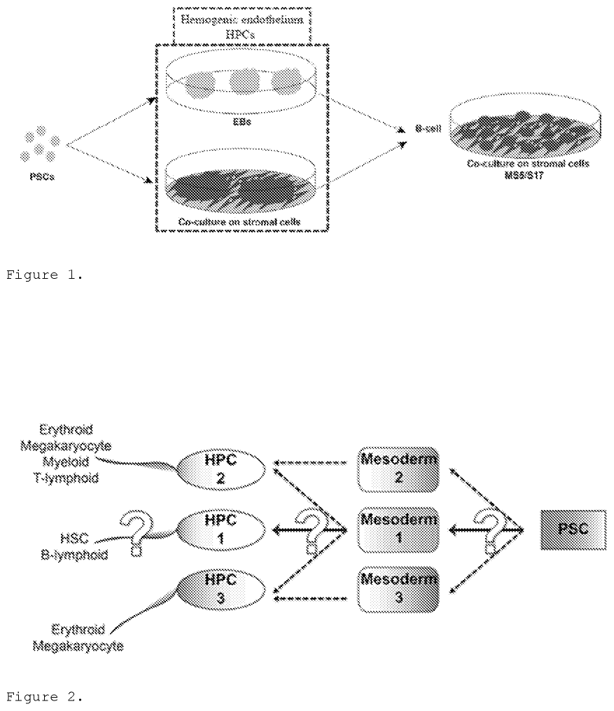

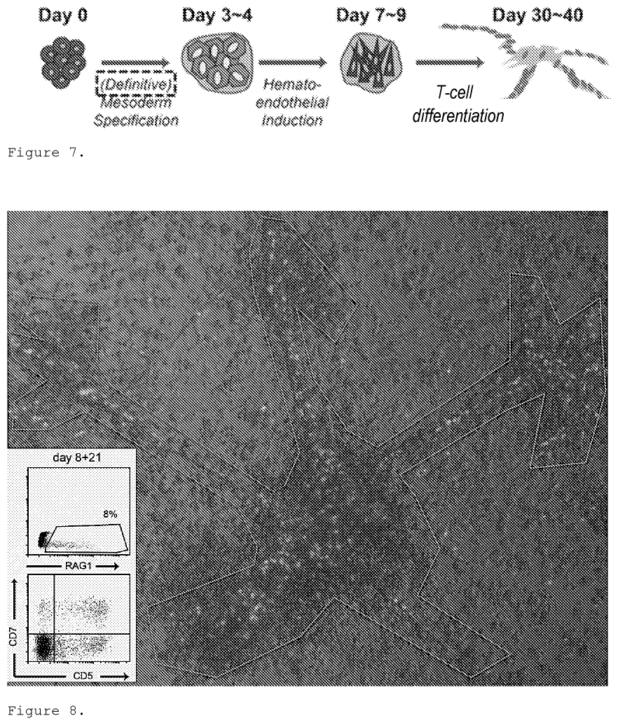

Overviews of current strategies for lymphocyte differentiation from PSCs to B-lymphocytes and T-lymphocytes are depicted in FIG. 1 and FIG. 7, respectively, and an overview of PSC differentiation models is depicted in FIG. 2.

The lymphopoietic characteristics of AGM-like structures developed during a definitive hematopoiesis differentiation protocol were investigated. The inventors found that T-lineage specification occurred in the absence of exogenous NOTCH1 ligands and without the need for purification of CD34⁺ progenitors by FACS. This finding enables a scalable method for generating T-cells for both research and for clinical applications.

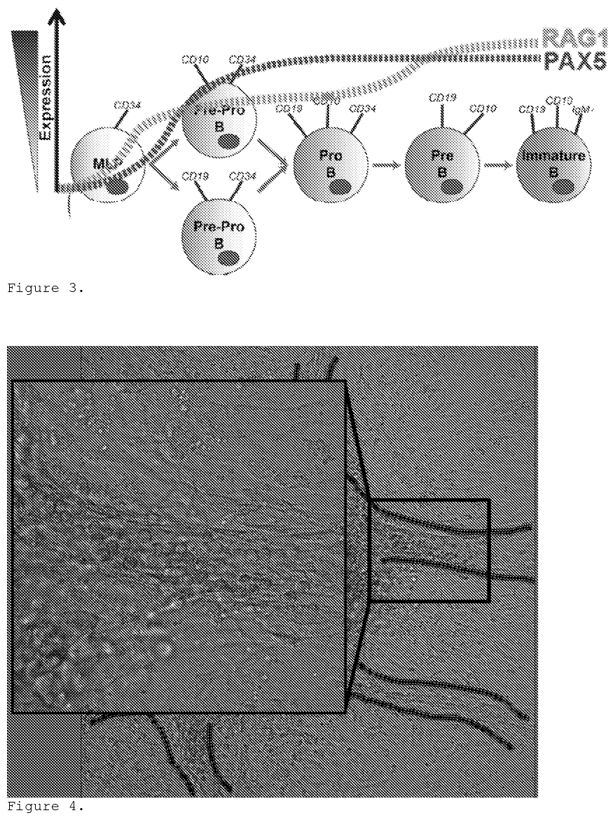

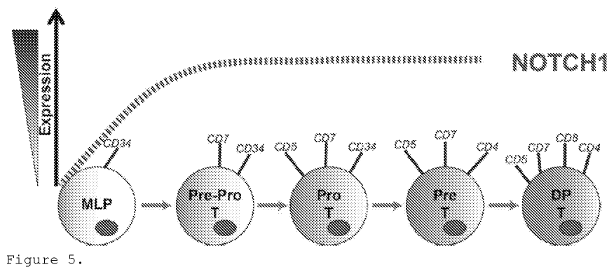

Overviews of B-lymphopoiesis and T-lymphopoiesis from a CD34⁺ cell are depicted in FIGS. 3 and 5, respectively. Generation of AGM from PSCs is illustrated in FIG. 4.

Having seen how AGM-like structures facilitated T-cell differentiation, B-lymphocyte differentiation from PSCs was investigated. Using growth factor directed differentiation, reproducible generation of B-lymphoid progenitors from multiple PSC lines was demonstrated.

Taken together, the invention provides robust and efficient methods for lymphocyte specification of PSCs. That is, the invention provides methods for producing a lymphocyte progenitor, and a lymphocyte progenitor produced according to those methods. In one embodiment, the lymphocyte progenitor is a T-cell progenitor. In one embodiment, the lymphocyte progenitor is a B-cell progenitor.

As used herein, “progenitor” refers to a cell in the hematopoietic lineage that is less differentiated than a cell that has differentiated down the lineage. It follows that T-cell progenitor will differentiate to a T-cell and a B-cell progenitor will differentiate to a B-cell.

T-Cell Progenitor

Analysis of newly formed hemogenic endothelium indicated that CD34⁺ endothelial cells expressed DLL4 and DLL1, two key ligands for NOTCH1. Exposure of early hematopoietic progenitors to either DLL1 or DLL4 diverted differentiation towards the T lymphoid lineages.

The commonly used hematopoietic supportive stromal cell line OP9 expresses both DLL1 and DLL4. Indeed, most current protocols for deriving T-cells from PSCs in vitro employ the OP9 system.

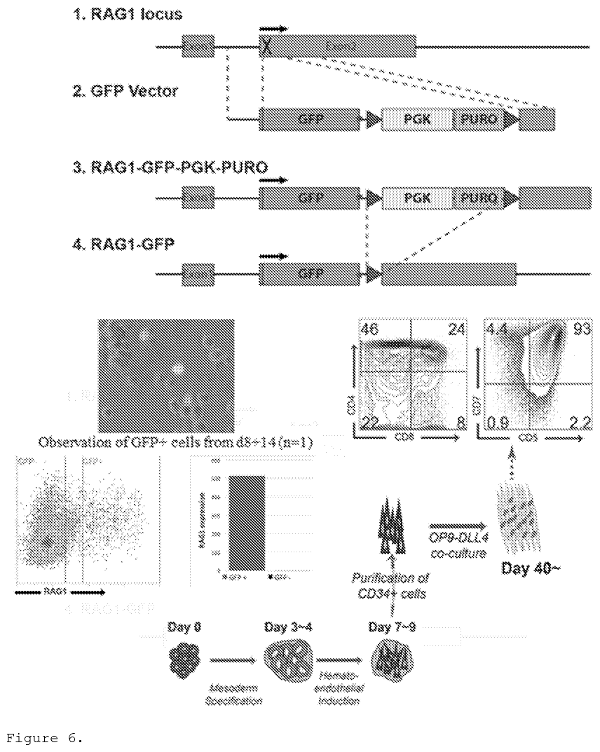

Expression of NOTCH ligands on AGM-like hemogenic endothelium (FIG. 4) prompted the inventors to ask whether such structures could initiate T-lymphoid differentiation. To help in the examination of this hypothesis, the inventors generated genetically modified human PSC lines in which sequences encoding GFP were targeted to the RAG1 locus, a gene that encodes one of the recombinases required for both T cell receptor and B-cell immunoglobulin gene rearrangements (FIG. 6).

Differentiation of PSC lines comprising this reporter gene using standard DLL4 OP9 co-cultures generated a population of GFP⁺ cells that expressed the early T-cell markers CD5 and CD7. To confirm the fidelity of this reporter gene, both GFP⁺ and GFP⁻ populations were isolated GFP detected by qPCR using a RAG1 specific probe. This analysis confirmed that RAG1 RNA expression was restricted to the GFP⁺ population (FIG. 6). Examination of cultures using fluorescence microscopy showed that RAG1+(GFP+) cells were easily visible, providing later opportunities for the direct observation of newly emerging RAG1+ cells (FIG. 6).

As noted above, AGM-like hemogenic endothelium expressed both DLL1 and DLL4, suggesting that under appropriate conditions, T lymphocyte development may occur directly in these cultures without stromal cell co-culture. Accordingly, differentiation experiments used either RAG1^{GFP/w} H9 human ESCs or RAG1^{GFP/w} RM3.5 iPSCs were conducted in which at day 8 of differentiation, embryoid bodies were transferred to adherent cultures and allowed to develop further in medium supplemented with FGF2, SCF, VEGF, and IL7. Under these conditions, vascular structures developed.

By day 21, RAG1⁺ cells associated with vascular like structures were observed (FIG. 8) and, with the aid of time lapse photography, observed RAG1+ cells moving along the vessels, consistently with their location within a lumen (not shown).

The cultures were harvested and cells labelled with antibodies that recognised the early T-cell markers CD5 and CD7 and the early B-lineage markers CD10 and CD19. No cells with a surface phenotype indicative of B lineage differentiation were detected. However, RAG1+ cells from these cultures expressed the T-lineage markers CD5 and CD7 (FIG. 8, inset). These results suggest that (a) appropriately competent progenitors arise during the differentiation of PSCs using the protocol disclosed herein and that (b) embryoid bodies generate an environment permissive and inductive for early T-cell commitment.

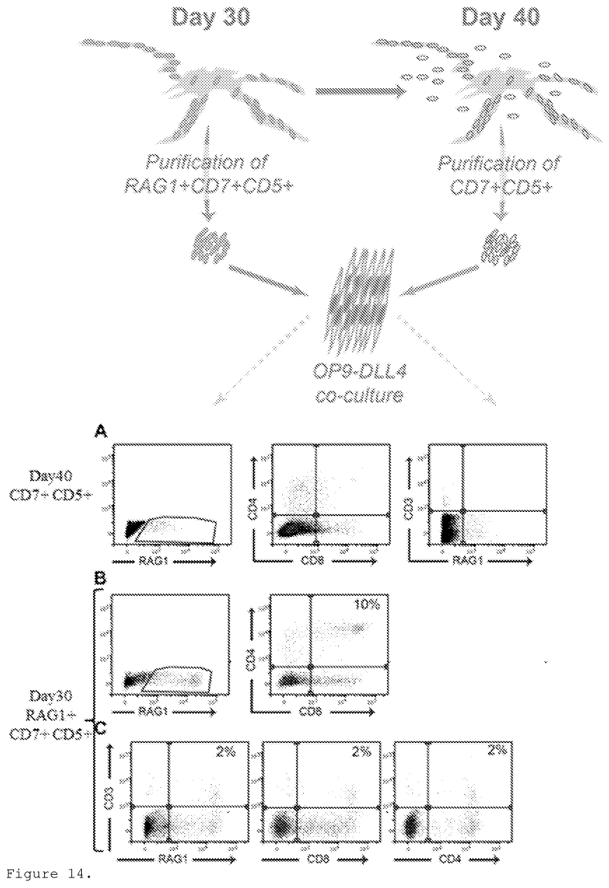

These cells did not progress to express CD4, indicating their development was arrested at the T-cell progenitor, or pro-T-cell, stage. In order to test whether this arrest was a property of the cells or the system in which they were cultured, RAG1⁺CD7⁺CD5⁺ pro-T-cells were transferred to the DLL4-OP9 stromal system. Within 7 days, RAG1^{high}CD3⁺CD4⁺CD8⁺ immature T-lymphocytes (2%) were detected by flow cytometry analysis (FIG. 14). In addition, a small fraction of the CD3⁺ cells were RAG1⁻CD4⁻CD8⁻, potentially representing γ/δ T lymphocytes. Interestingly, when this same experiment was conducted with developmentally arrested RAG1⁻CD7⁺CD5⁺ pro-T-cells, no up-regulation of later T-lineage differentiation markers was observed. These experiments indicated there is a window of opportunity for pro-T-cells to progress further along the T-lineage.

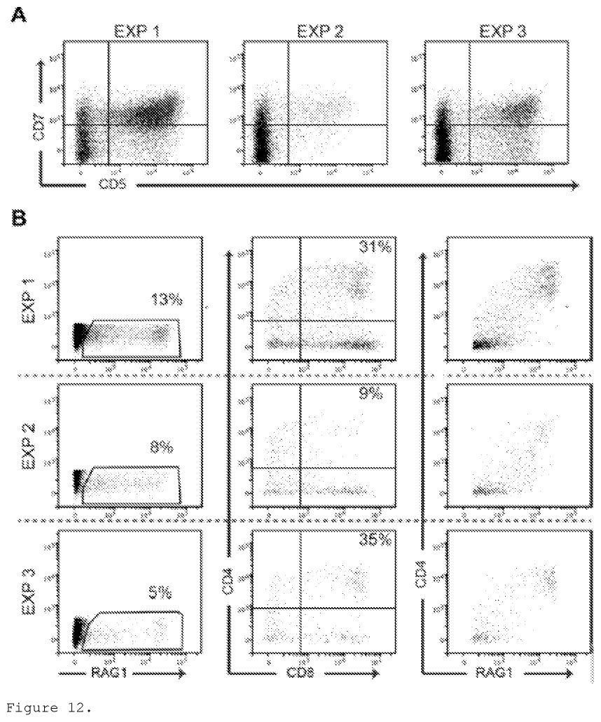

In summary, culturing day 8 embryoid bodies at an ALI increased the proportion of major vessels in the AGM, the location of cells thought to be the major source of DLL4. Analysis of T-cell differentiation within these cultures indicated that this modification dramatically increased the frequency of CD5⁺CD7⁺ T-cell progenitors and enabled a fraction of cells to reach the CD4⁺CD8⁺ stage (FIG. 11 and FIG. 12).

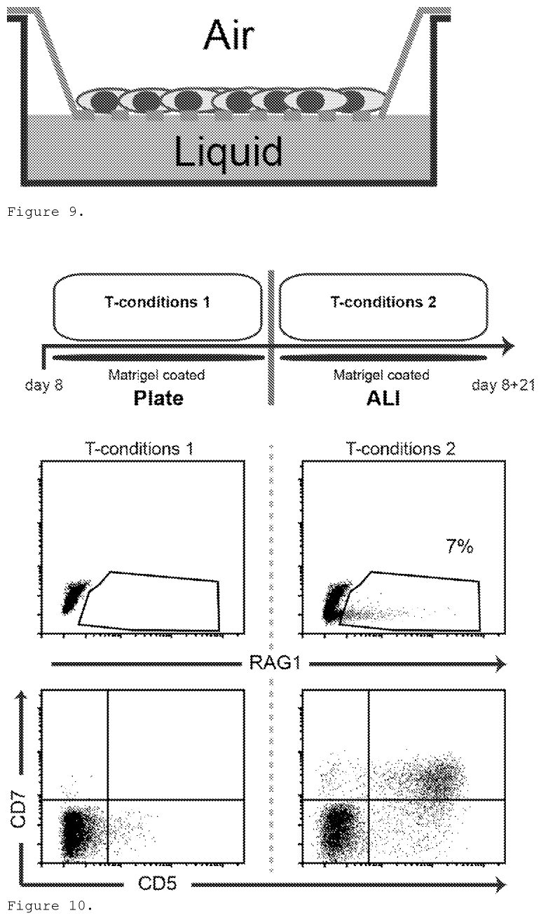

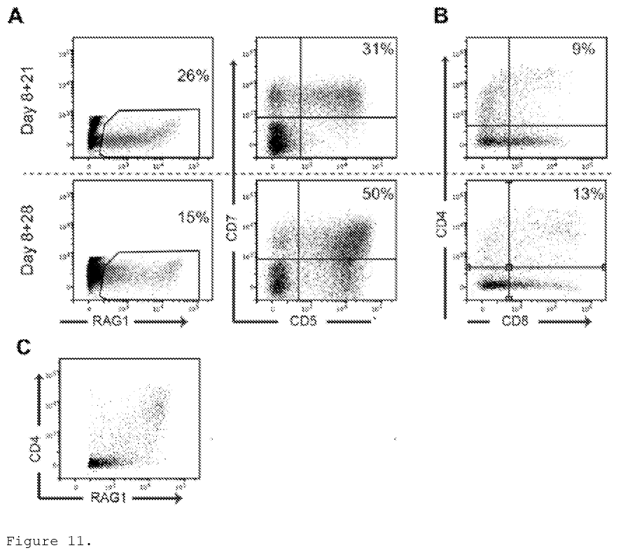

A schematic representation of T-lymphopoiesis from PSCs using ALI is depicted in FIG. 9. A schematic representation and flow cytometry plots of T-lymphopoiesis from PSCs using ALI culture showing that ALI is a superior platform for generating T-cells are depicted in FIG. 10.

Therefore, the inventors have developed a xeno-free system for generating T-cell progenitors from PSCs. Importantly, generation of cells under these conditions disclosed herein does not require purification of CD34⁺ progenitors, making it amenable for commercial scale up.

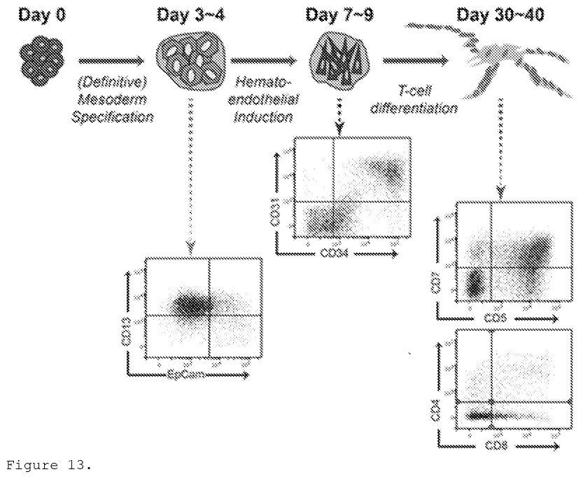

In short, the inventors have shown that T-cell progenitors can be derived from PSCs without the need for either cell sorting or mouse feeder layers. A summary of T-lymphopoiesis from PSCs according to the present disclosure is depicted in FIG. 13.

Clinical applications of T-cell progenitors. CAR T-cells have attracted much attention as therapies for malignancies in which a tumour specific antigen is known. Current methods of CAR-T cell production require the transduction of patient derived T-cells with lentiviral vectors encoding the CAR. Others have reported the generation of CAR-T-cells from iPSCs, with the CAR again delivered using non-targeted integration of lentiviral vectors. The present invention enable preparation of validated CAR-T cells to be produced in vitro from genetically modified iPSCs sourced from established iPSC haplobanks. The advantage of using an off the shelf CAR-T cell would be that its characteristics would be well documented and its performance easily monitored. Additionally, using iPSCs as a starting cell affords the opportunity to engineer in additional safeguards (such as suicide switches) should they be deemed necessary.

More broadly, however, the present disclosure extends to any T cell with defined antigen specificity, for example carrying a naturally-occurring T-cell receptor isolated from a subject.

In addition, the methods may be used to generate T-cells comprising disease-specific mutations for drug screening, for example.

B-Cell Progenitor

Few methods for the generation of B-cells from PSCs have been reported, and, those described thus far have gained little traction within the field. One potential road block for generation of B-lineage cells relates to the developmental status of progenitors capable of B lymphopoiesis. During mouse development, blood cells are generated in successive waves that arise at distinct sites within the embryo. Within the mouse embryo, the first wave of primitive blood cells arise in the yolk sac and yield mainly nucleated erythroid cells and a limited repertoire of myeloid cells. A second wave of haematopoiesis produces more mature erythroid cells, myeloid cells including macrophages, megakaryocytes, neutrophils and mast cells. In addition, this wave of blood cell development can also generate T-lineage cells. The final wave of blood cell development, originating in the AGM region, includes progenitors capable of generating all cell types found in the adult blood system, including repopulation competent haematopoietic stem cells (HSCs).

Studies of mouse hematopoiesis suggest that lymphoid restricted progenitors emerge from endothelial cells before, and independent of, the generation of HSCs. These pre-HSC lymphoid cells have restricted potential, generating innate B-1 and marginal zone B cells (no B-2 cells) and all types of T-cells. However, in humans, B/T-lymphocytes have not been identified until 8-9 weeks of gestation, well after the emergence of HSCs in the AGM (5-6 weeks). Taken together, these results suggest that unlike in mice, human B-cells (and not T-cells) might only emerge from HSCs.

In light of the above, the appearance of AGM-like structures within the PSC differentiation experiments disclosed herein prompted the inventors to ask if these cultures might contain cells with B-lymphoid potential.

To this end, RAG1^{GFP/w} iPSCs were differentiated towards AGM-like hemogenic endothelium. At differentiation day 15, CD34⁺43⁺ and CD34⁺43⁻ populations were isolated by FACS and subsequently seeded onto confluent OP9-DLL4 stromal cell cultures containing T-cell medium (Controls: Factors: SCF, FLT3L, IL7) or B-cell medium (Factors: SCF, FLT3L, IL3, SDF1 and the NOTCH signalling inhibitor, DAPT). Under these conditions, control cultures generated T-cell progenitors identified by expression of RAG1, CD7 and CD5. In cultures containing B-cell medium, a low but meaningful number of RAG1⁺, CD10⁺ and CD19⁺ B-lymphoid progenitors were observed. The CD10⁺CD19⁺ committed B-cell progenitors expressed a low level of CD34, possibly indicating they represented cells in progression from the pro-B-cell to pre-B-cell stage. Moreover, a population of RAG1⁺CD10⁺CD19⁻ pre-pro-B-cells, which require RAG1 expression for DJ rearrangement of the immunoglobulin heavy chain, was also observed.

As described above, it was consistently observed that B-cell lineage commitment was induced when HPCs were cultured in medium supplemented with DAPT and SCF and co-cultured on OP9-DLL4 cells. In these cultures, DAPT was included to block or attenuate NOTCH1 signalling mediated by DLL4 ligands whilst SCF was included because of its documented proliferative effect on HPCs and B-lymphocytes.

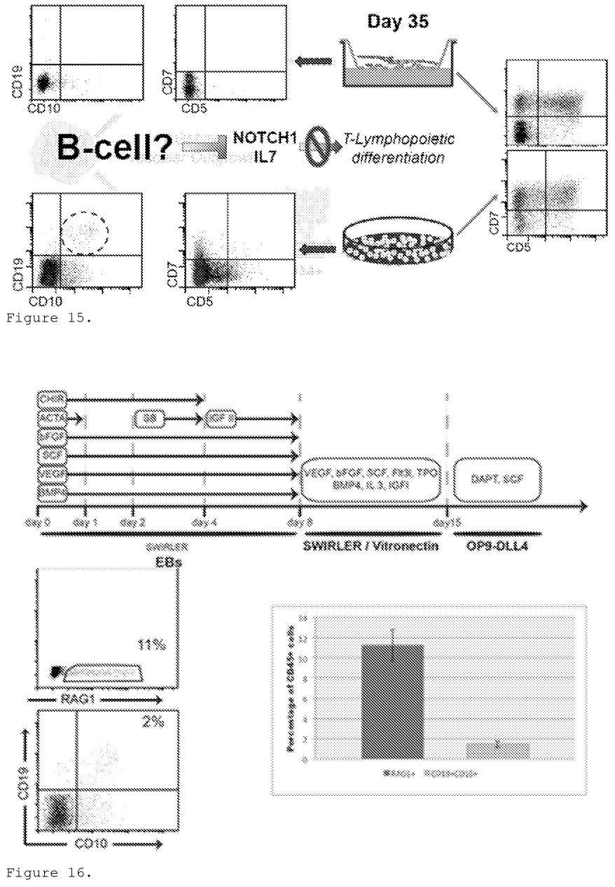

A schematic representation B-lymphopoiesis via blocking T-lymphopoiesis is depicted in FIG. 15.

Nevertheless, the inventors' strategy was to ensure that, as far as possible, the HPCs/HSCs in culture were restricted from becoming anything but B-lymphocytes. However, this method proved to be insufficient for robust B-cell differentiation because (a) only 1-2% B-cells could be generated and (b) the remaining cells in culture rapidly acquired T-cell fate when the DAPT was removed from the media from day 23 (data not shown). Importantly, attempts to apply this protocol to H9 hESCs yielded no B-lineage cells.

In order to simplify the method, the consequence of not purifying CD34⁺ HPCs was examined. Instead, intact embryoid bodies were transferred onto OP9-DLL4 stromal cells at around day 15. This approach had the advantage of retaining high viability of the cultures which contrasted with significant cell loss associated with disaggregating cultures in preparation for FACS.

In addition, it was also speculated that the endogenous signalling produced by different cell types in the embryoid bodies might prompt lymphocyte induction or potentially assist the survival or maturation of early B-cell progenitors.

To test this, RAG1^{GFP/w} iPSCs were differentiated until day 15 and then transferred onto a confluent layer of OP9-DLL4 stromal cells and medium supplemented with DAPT and SCF. After more than 2 weeks of differentiation (passaged after 1 week), the culture was analysed for the presence of B-cell progenitors. At this stage, nearly 4% of CD45⁺ hematopoietic cells expressed RAG1, CD10 and CD19. This result compared favourably with the average of 1% obtained for this population when purified day 15 CD34⁺ cells were used.

A schematic representation and flow cytometry plots showing one example of B-lymphopoiesis according to the disclosure are depicted in FIG. 16.

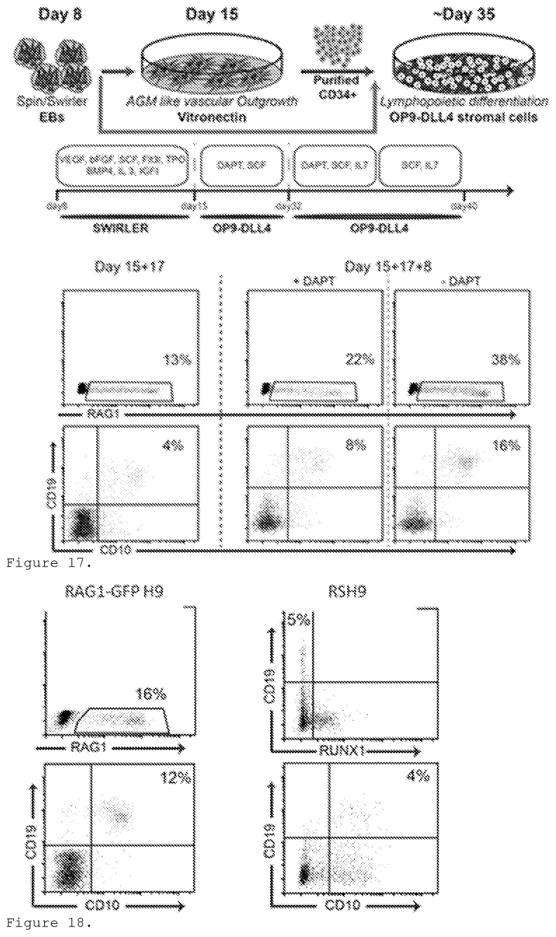

Further differentiation of these cells for another 8 days in presence of SCF and IL7 resulted in an increased proportion of pre-B-cells. At this stage, the de-repression of NOTCH1 signalling allowed by removal of DAPT appeared to have a positive effect on the frequency of B-lineage cells, with flow cytometry analysis indicating up to 16% of CD45⁺ cells were CD10⁺CD19⁺RAG1⁺ pre-B-cells (FIG. 17).

In order to further assess the general applicability of this differentiation regime, this protocol was applied to two different cell lines, RAG1^{GFP/w} H9 and RUNX1c^{GFP/w} SOX17^{mCherry/w} H9 (RSH9), both derivatives of H9 hESCs. Although RSH9 line robustly develops AGM-like structures that give rise to hematopoietic progenitors, previous attempts at obtaining B-lineage cells from FACS purified day 15 CD34⁺ progenitors were unsuccessful. Encouragingly, after 2 weeks of co-culture, a small population of RAG1⁺CD10⁺CD19⁺ cells in cultures of the RAG1^{GFP/w} H9 cells was detected, and CD10⁺CD19⁺ cells in the cultures representing the RSH9 line (FIG. 18). The preliminary data show that B-cell progenitors can be generated from AGM stage cultures without the need for an intermediate sorting step.

Clinical applications of B-cell progenitors. Humanised antibodies directed at a range of clinically relevant targets are a major development in a raft of new therapeutic agents. The successful and robust derivation of human antibody producing B-cells would provide an avenue to antibody generation that circumvented current pipelines that transfer the antigen specificity of rodent derived immunoglobulins to human antibody backbones. In addition, methods for producing B-cells (and T-cells) in vitro will enable the construction of models of human lymphoid disease and provide a platform for drug screening in a clinically relevant human setting.

Methods of antibody production are well known to the person skilled in the art.

Otherwise, the methods may be used to generate B-cells comprising disease-specific mutations for drug screening, for example.

Unless defined otherwise in this specification, technical and scientific terms used herein have the same meaning as commonly understood by the person skilled in the art to which this invention belongs and by reference to published texts.

It is to be noted that the term “a” or “an” refers to one or more, for example, “a molecule,” is understood to represent one or more molecules. As such, the terms “a” or “an”, “one or more,” and “at least one” may be used interchangeably herein.

In the claims which follow and in the description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

The term “about” as used herein contemplates a range of values for a given number of ±25% the magnitude of that number. In other embodiments, the term “about” contemplates a range of values for a given number of ±20%, ±15%, ±10%, or ±5% the magnitude of that number. For example, in one embodiment, “about 3 μM” indicates a value of 2.7 to 3.3 μM (i.e. 3 μM±10%), and the like.

Similarly, while differentiation processes include ordered, sequential events, the timing of the events may be varied by at least 25%. For example, while a particular step may be disclosed in one embodiment as lasting one day, the event may last for more or less than one day. For example, “one day” may include a period of about 18 to about 30 hours. In other embodiments, periods of time may vary by ±20%, ±15%, ±10%, or ±5% of that period of time. Periods of time indicated that are multiple day periods may be multiples of “one day,” such as, for example, two days may span a period of about 36 to about 60 hours, and the like. In another embodiment, time variation may be lessened, for example, where day 2 is 48±3 hours from day 0; day 4 is 96±3 hours from day 0, and day 5 is 120 hours±3 hours from day 0.

As used herein, about 2 days may be 1.5, 2 or 2.5 days, about 4 days may be 3.5, 4 or 4.5 days, about 8 days may be 6, 6.5, 7, 7.5, 8, 8.5, 9, 9.5 or 10 days, about 15 days may be 12, 12.5, 13, 13.5, 14, 14.5, 15, 15.5, 16, 16.5, 17, 17.5 or 18 days, about 22 days may be 18 days to 18, 18.5, 19, 19.5, 20, 20.5, 21, 21.5, 22, 22.5, 23, 23.5, 24, 24.5, 25, 25.5 or 26 days, about 29 days may be 25, 25.5, 26, 26.5, 27, 27.5, 28, 28.5, 29, 29.5, 30, 30.5, 31, 31.5, 32, 32.5, or 33 days, and about 32 days may be 28, 28.5, 29.5, 30, 30.5, 31, 31.5, 32, 32.5, 33, 33.5, 34, 34.5, 35, 35.5 or 36 days.

As used herein, 0 to 2 days may be any combination of 0.5 days to 1.5, 2 or 2.5 days. Two to 4 days may be any combination of 1.5, 2 or 2.5 days to 3.5, 4 or 4.5 days. Four to 8 days may be any combination of 3, 3.5, 4, 4.5 or 5 days to 6, 6.5, 7, 7.5, 8, 8.5, 9, 9.5 or 10 days. Eight to 15 days may be any combination of 6, 6.5, 7, 7.5, 8, 8.5, 9, 9.5 or 10 days to 12, 12.5, 13, 13.5, 14, 14.5, 15, 15.5, 16, 16.5, 17, 17.5 or 18 days. Fifteen to 22 days may be any combination of 12, 12.5, 13, 13.5, 14, 14.5, 15, 15.5, 16, 16.5, 17, 17.5 or 18 days to 18, 18.5, 19, 19.5, 20, 20.5, 21, 21.5, 22, 22.5, 23, 23.5, 24, 24.5, 25, 25.5 or 26 days. Twenty-two to 29 days may be 18 days to 18, 18.5, 19, 19.5, 20, 20.5, 21, 21.5, 22, 22.5, 23, 23.5, 24, 24.5, 25, 25.5 or 26 days to 25, 25.5, 26, 26.5, 27, 27.5, 28, 28.5, 29.5, 30, 30.5, 31, 31.5, 32, 32.5 or 33 days.

In one embodiment of any one of the first to third aspects, the lymphocyte progenitor is a T-cell progenitor, and the method comprises culturing a PSC-derived CD34⁺ PSC at an ALI in a medium comprising vascular endothelial growth factor (VEGF), basic fibroblast growth factor (bFGF, FGF2), stem cell factor (SCF), fms-related tyrosine kinase 3 ligand (FLT3L), and interleukin-7 (IL-7). In embodiments, the medium may further comprise IL-3 and/or BMP4.

In one embodiment of any one of the first to third aspects, the lymphocyte progenitor is a T-cell progenitor, and the medium at each of about day 8 to about day 15, about day 15 to about day 22, and about day 22 onward comprises about 5, 10, 15, 20, 25, 30, 35, 40, 45, 50, 55, 60, 65, 70, 75, 80, 85, 90, 95 or 100 ng/mL VEGF. In one embodiment, at about day 22 onward, the medium comprises less VEGF than at about day 8 to about day 15 and about day 15 to about day 22 and comprises about 5, 10, 15, 20, 25, 30, 35, 40, 45 or 50 ng/mL VEGF. In one embodiment, the medium at each of about day 8 to about day 15, about day 15 to about day 22, and about day 22 onward comprises about 50 ng/mL VEGF.

In one embodiment of any one of the first to third aspects, the lymphocyte progenitor is a T-cell progenitor, and the medium at each of about day 8 to about day 15, about day 15 to about day 22, and about day 22 onward comprises about 1, 2, 3, 4, 5, 6, 7, 8, 9, 10, 11, 12, 13, 14, 15, 16, 17, 18, 19 or 20 ng/mL FGF2. In one embodiment, at about day 15 to about day 22 and at about day 22 onward, the medium comprises less FGF2 that at about day 8 to about day 15 or may be omitted. In one embodiment, the medium at each of about day 8 to about day 15, about day 15 to about day 22, and about day 22 onward comprises about 10 ng/mL FGF2. In one embodiment, the medium at about day 8 to about day 15 comprises about 10 ng/mL FGF2, and at about day 15 to about day 22 and about day 22 onward FGF2 is omitted.

In one embodiment of any one of the first to third aspects, the lymphocyte progenitor is a T-cell progenitor, and the medium at about day 8 to about day 15 comprises about 20, 25, 30, 35, 40, 45, 50, 55, 60, 65, 70, 75, 80, 85, 90, 95, 100, 105, 110, 115, 120, 125, 130, 135, 140, 145 or 150 ng/mL SCF. In one embodiment, at about day 15 to about day 22, the medium comprises less SCF than at day 8 to day 15 and comprises about 5, 10, 15, 20, 25, 30, 35, 40, 45, 50, 55, 60, 65, 70 or 75 ng/mL SCF. In one embodiment, at about day 22 onward, the medium comprises less SCF than at about day 15 to about day 22 and comprises about 5, 10, 15, 20, 25, 30, 35, 40, 45 or 50 ng/mL SCF or may be omitted. In one embodiment, the medium at about day 8 to about day 15 comprises about 100 ng/mL SCF, at about day 15 to about day 22 comprises about 50 ng/mL SCF, and at about day 22 onward comprises about 25 ng/mL SCF. In another embodiment, the medium at about day 8 to about day 15 comprises about 100 ng/mL SCF, at about day 15 to about day 22 comprises about 20 ng/mL SCF, and at about day 22 onward SCF is omitted.

In one embodiment of any one of the first to third aspects, the lymphocyte progenitor is a T-cell progenitor, and the medium at each of about day 8 to about day 15, about day 15 to about day 22, and about day 22 onward comprises about 1, 2, 3, 4, 5, 6, 7, 8, 9, 10, 11, 12, 13, 14, 15, 16, 17, 18, 19 or 20 ng/mL FLT3L. In one embodiment, at about day 15 to about day 22 and at about day 22 onward, the medium comprises less FLT3L that at about day 8 to about day 15 or may be omitted. In one embodiment, the medium at each of about day 8 to about day 15, about day 15 to about day 22, and about day 22 onward comprises about 10 ng/mL FLT3L. In another embodiment, the medium at about day 8 to about day 15 comprises about 10 ng/mL FLT3L, and at about day 15 to about day 22 and about day 22 onward FLT3L is omitted.

In one embodiment of any one of the first to third aspects, the lymphocyte progenitor is a T-cell progenitor, and the medium may further comprise IL-3. In one embodiment, the medium at each of about day 8 to about day 15 and about day 15 to about day 22 comprises about 5, 10, 15, 20, 25, 30, 35 or 40 ng/mL IL-3. In one embodiment, at about day 15 to about day 22 or at about day 22 onward, the medium comprises less IL-3 than at about day 8 to about day 15 or about day 15 to about day 22, respectively, and comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 ng/mL IL-3, or IL-3 may be omitted. In one embodiment, the medium at each of about day 8 to about day 15 and about day 15 to about day 22 comprises 20 ng/mL IL-3, and at about day 22 onward comprises about 10 ng/mL IL-3. In another embodiment, the medium at about day 8 to about day 15 comprises 10 ng/mL IL-3, and at each of about day 15 to about day 22 and about day 22 onward IL-3 is omitted.

In one embodiment of any one of the first to third aspects, the lymphocyte progenitor is a T-cell progenitor, and the medium may further comprise BMP-4. In one embodiment, the medium at any or all of about day 8 to about day 15, about day 15 to about day 22, and about day 22 onward comprises about 1, 2, 3, 4, 5, 6, 7, 8, 9, 10, 11, 12, 13, 14, 15, 16, 17, 18, 19 or 20 ng/mL BMP4. In one embodiment, the medium at each of about day 8 to about day 15, about day 15 to about day 22, and about day 22 onward comprises about 10 ng/mL BMP4.

In one embodiment of any one of the first to third aspects, the lymphocyte progenitor is a T-cell progenitor, and the medium may further comprise IL-6. In one embodiment, the medium at any or all of about day 8 to about day 15, about day 15 to about day 22, and about day 22 onward comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 ng/mL IL-6. In one embodiment, the medium at each of about day 8 to about day 15 and about day 15 to about day 22 comprises about 20 ng/mL IL-6, and at about day 22 onward comprises about 10 ng/mL IL-6.

In one embodiment of any one of the first to third aspects, the lymphocyte progenitor is a T-cell progenitor, and the medium may further comprise IL-2. In one embodiment, the medium at any or all of about day 8 to about day 15, about day 15 to about day 22, and about day 22 onward comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 U/mL IL-2. In one embodiment, the medium at each of about day 8 to about day 15, about day 15 to about day 22, and about day 22 onward comprises about 20 U/mL IL-2.

In an embodiment of any one of the first to third aspects when producing a T-cell progenitor, culturing at the ALI is for about 1, 1.5, 2, 2.5, 3, 3.5, 4, 4.5, 5, 5.5, 6, 6.5, 7, 7.5, 8, 8.5, 9, 9.5 or 10 weeks. In one embodiment, culturing at the ALI is for about 2 weeks to about 5 weeks.

In one embodiment of the first to third aspect, the lymphocyte progenitor is a B-cell progenitor, and the method comprises culturing the CD34⁺ cell in a medium comprising a NOTCH1 inhibitor during or after culturing the CD34⁺ cell at the ALI. In embodiments, the medium may further comprise IL-7, FLT3L, TPO, IL-3, SDF1 and/or GCSF.

In one embodiment of the first to third aspect, the lymphocyte progenitor is a B-cell progenitor, and the method comprises culturing the CD34⁺ cell in a medium comprising a NOTCH1 inhibitor from any combination of about day 12, 13, 14, 15, 16, 17 or 18 to about day 18, 19, 20, 21, 22, 23 or 24 during or after culturing the CD34⁺ cell at the ALI. In one embodiment, the method comprises culturing the CD34⁺ cell in a medium comprising a NOTCH1 inhibitor from about day 15 to about day 21 or about day 22 during or after culturing the CD34⁺ cell at the ALI. In one embodiment, the medium comprises about 1, 2, 3, 4, 5, 6, 7, 8, 9, 10, 11, 12, 13, 14, 15, 16, 17, 18, 19 or 20 μM NOTCH1 inhibitor. In one embodiment, at about day 15 to about day 21 or about day 22, the medium comprises about 10 μM NOTCH1 inhibitor.

In one embodiment of the first to third aspect, the lymphocyte progenitor is a B-cell progenitor, and the medium at about day 21 or about day 22 to about day 29 comprises about 20, 25, 30, 35, 40, 45, 50, 55, 60, 65, 70, 75, 80, 85, 90, 95, 100, 105, 110, 115, 120, 125, 130, 135, 140, 145 or 150 ng/mL CD117 activator. In one embodiment, at about day 21 or about day 22 to about day 29, the medium comprises about 5, 10, 15, 20, 25, 30, 35, 40, 45, 50, 55, 60, 65, 70 or 75 ng/mL CD117 activator. In one embodiment, at about day 29 onward, the medium comprises less CD117 activator than at about day 21 or about day 22 to about day 29 and comprises about 5, 10, 15, 20, 25, 30, 35, 40, 45 or 50 ng/mL CD117 activator. In one embodiment, the medium at about day 21 or about day 22 to about day 29 comprises about 50 ng/mL CD117 activator, and at about day 29 onward comprises about 20 ng/mL CD117 activator. In one embodiment, the method comprises culturing the CD34⁺ cell in a medium comprising a CD117 activator from about day 22. In one embodiment, the method comprises culturing the CD34⁺ cell in a medium comprising a CD117 activator from about day 22 for about 14, 15, 16, 17, 18, 19 or 20, 21, 22, 23, 24, 25, 26, 27 or 28 days. In one embodiment, the method comprises culturing the CD34⁺ cell in a medium comprising a CD117 activator from about day 22 for about 18 days.

In one embodiment of any one of the fourth to sixth aspects, the method comprises: (i) co-culturing a PSC-derived CD34⁺ cell and a stromal cell in a medium comprising a CD117 activator and a NOTCH1 inhibitor; and (ii) co-culturing the cells of (i) in a medium comprising interleukin-7 (IL-7). In one embodiment, the method comprises culturing the CD34⁺ cell in a medium comprising a NOTCH1 inhibitor from about day 12, 13, 14, 15, 16, 17 or 18 onwards. In one embodiment, the method comprises culturing the CD34⁺ cell in a medium comprising a NOTCH1 inhibitor up to about day 29, 30, 31, 32, 33, 34, 35, 36, 37, 38, 39, 40, 41 or 42. In one embodiment, the medium comprises about 1, 2, 3, 4, 5, 6, 7, 8, 9, 10, 11, 12, 13, 14, 15, 16, 17, 18, 19 or 20 μM NOTCH1 inhibitor. In one embodiment, the medium comprises about 10 μM NOTCH1 inhibitor. In one embodiment, at any or all of about day 15 to about day 22, about day 22 to about day 29, and about day 29 onwards, the medium comprises about 1, 2, 3, 4, 5, 6, 7, 8, 9, 10, 11, 12, 13, 14, 15, 16, 17, 18, 19 or 20 μM NOTCH1 inhibitor. In one embodiment, at any or all of about day 15 to about day 22, about day 22 to about day 29, and about day 29 onwards, the medium comprises about 10 μM NOTCH1 inhibitor. In one embodiment, at each of about day 15 to about day 22, about day 22 to about day 29, and about day 29 onwards, the medium comprises about 10 μM NOTCH1 inhibitor.

In one embodiment of any one of the fourth to sixth aspects, the method comprises: (i) co-culturing a PSC-derived CD34⁺ cell and a stromal cell in a medium comprising a CD117 activator and a NOTCH1 inhibitor; and (ii) co-culturing the cells of (i) in a medium comprising interleukin-7 (IL-7). In one embodiment, the method comprises culturing the CD34⁺ cell in a medium comprising a CD117 activator from about day 12, 13, 14, 15, 16, 17 or 18 onwards. In one embodiment, the method comprises culturing the CD34⁺ cell in a medium comprising a CD117 activator up to about day 29, 30, 31, 32, 33, 34, 35, 36, 37, 38, 39, 40, 41, 42 or 43. In one embodiment, the medium comprises about 20, 25, 30, 35, 40, 45, 50, 55, 60, 65, 70, 75, 80, 85, 90, 95, 100, 105, 110, 115, 120, 125, 130, 135, 140, 145 or 150 ng/mL CD117 activator. In one embodiment, the medium comprises about 100, 50, or 20 ng/mL CD117 activator. In one embodiment, at about day 22 to about day 29, the medium comprises less CD117 activator than at day 15 to day 22 and comprises about 5, 10, 15, 20, 25, 30, 35, 40, 45, 50, 55, 60, 65, 70 or 75 ng/mL CD117 activator. In one embodiment, at about day 29 onward, the medium comprises less CD117 activator than at about day 22 to about day 29 and comprises about 5, 10, 15, 20, 25, 30, 35, 40, 45 or 50 ng/mL CD117 activator. In one embodiment, the medium at about day 15 to about day 22 comprises about 100 ng/mL CD117 activator, at about day 22 to about day 29 comprises about 50 ng/mL CD117 activator, and at about day 29 onward comprises about 20 ng/mL CD117 activator. In one embodiment, the method comprises culturing the CD34⁺ cell in a medium comprising a CD117 activator from about day 15. In one embodiment, the method comprises culturing the CD34⁺ cell in a medium comprising a CD117 activator from about day 15 for about 14, 15, 16, 17, 18, 19, 20, 21, 22, 23, 24, 25, 26, 27 or 28 days. In one embodiment, the method comprises culturing the CD34⁺ cell in a medium comprising a CD117 activator from about day 15 for about 25 days.

In one embodiment of any one of the fourth to sixth aspects, the method comprises co-culturing the cells in a medium comprising a CD117 activator and a NOTCH1 inhibitor from about day 15 for about 14, 15, 16, 17, 18, 19, 20, 21, 22, 23, 24, 25, 26, 27 or 28 days.

In one embodiment of any one of the fourth to sixth aspects, the method comprises co-culturing the cells in a medium comprising a CD117 activator and a NOTCH1 inhibitor from about day 15 for about 15, 16, 17, 18, 19 or 20 days, then co-culturing the cells in a medium comprising a CD117 activator, a NOTCH1 inhibitor and IL-7 for about 1, 2, 3, 4, 5, 6, 7 or 8 days, then co-culturing the cells in a medium comprising a CD117 activator and IL-7 for about 1, 2, 3, 4, 5, 6, 7 or 8 days.

In one embodiment of the first to sixth aspects, the lymphocyte progenitor is a B-cell progenitor, and the method comprises culturing in a medium comprising IL-7. In one embodiment, the medium at about day 15 to about day 22 and at about day 22 to day 29, comprises about 1, 2, 3, 4, 5, 6, 7, 8, 9, 10, 11, 12, 13, 14, 15, 16, 17, 18, 19 or 20 ng/mL IL-7, and at about day 29 onward comprises about 1, 2, 3, 4, 5, 6, 7, 8, 9 or 10 ng/mL IL-7. In one embodiment, the medium at about day 15 to about day 22 and at about day 22 to day 29, comprises about 10 ng/mL IL-7, and at about day 29 onward comprises about 5 ng/mL IL-7. In another embodiment, at about day 32 onward, the medium comprises about 1, 2, 3, 4, 5, 6, 7, 8, 9, 10, 11, 12, 13, 14, 15, 16, 17, 18, 19 or 20 ng/mL IL-7, and the method comprises culturing for about 5, 6, 7, 8, 9, 10 or 11 days.

In one embodiment of any one of the first to sixth aspects, the lymphocyte progenitor is a B-cell progenitor, and the medium at each of about day 15 to about day 22, about day 22 to about day 29, and about day 29 onward comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 ng/mL FLT3L. In one embodiment, at about day 22 to about day 29 and at about day 29 onward, the medium comprises less FLT3L that at about day 15 to about day 22. In one embodiment, the medium at about day 15 to about day 22 comprises about 20 ng/mL FLT3L, and at each of about day 22 to about day 29 and about day 29 onward the medium comprises about 10 ng/mL FLT3L.

In one embodiment of any one of the first to sixth aspects, the lymphocyte progenitor is a B-cell progenitor, and the medium at each of about day 15 to about day 22, about day 22 to about day 29, and about day 29 onward comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 ng/mL FLT3L. In one embodiment, at about day 22 to about day 29 and at about day 29 onward, the medium comprises less FLT3L that at about day 15 to about day 22. In one embodiment, the medium at about day 15 to about day 22 comprises about 20 ng/mL FLT3L, and at each of about day 22 to about day 29 and about day 29 onward the medium comprises about 10 ng/mL FLT3L.

In one embodiment of any one of the first to sixth aspects, the lymphocyte progenitor is a B-cell progenitor, and the medium may further comprise TPO. In one embodiment, the medium at each of about day 15 to about day 22 and about day 22 to about day 29 comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 ng/mL TPO. In one embodiment, at about day 29 onward, the medium comprises less TPO than at about day 15 to about day 22 or about day 22 to about day 29, or TPO may be omitted. In one embodiment, the medium at about day 15 to about day 22 comprises about 20 ng/mL TPO, at about day 22 to about day 29 comprises about 10 ng/mL TPO, and at about day 29 onward TPO is omitted.

In one embodiment of any one of the first to sixth aspects, the lymphocyte progenitor is a B-cell progenitor, and the medium may further comprise SDF1. In one embodiment, the medium at each of about day 15 to about day 22 and about day 22 to about day 29 comprises about 10, 20, 30, 40, 50, 60, 70, 80, 90, 100, 110, 120, 130, 140, 150, 160, 170, 180, 190 or 200 ng/mL SDF1. In one embodiment, at about day 29 onward, the medium comprises less SDF1 than at about day 15 to about day 22 or about day 22 to about day 29, or SDF1 may be omitted. In one embodiment, the medium at each of about day 15 to about day 22 and about day 22 to about day 29 comprises about 100 ng/mL SDF1, and at about day 29 onward SDF1 is omitted.

In one embodiment of any one of the first to sixth aspects, the lymphocyte progenitor is a B-cell progenitor, and the medium may further comprise GCSF. In one embodiment, the medium at each of about day 15 to about day 22 and about day 22 to about day 29 comprises about 10, 20, 30, 40, 50, 60, 70, 80, 90, 100, 110, 120, 130, 140, 150, 160, 170, 180, 190 or 200 ng/mL GCSF. In one embodiment, at about day 29 onward, the medium comprises less GCSF than at about day 15 to about day 22 or about day 22 to about day 29, or GCSF may be omitted. In one embodiment, the medium at each of about day 15 to about day 22 and about day 22 to about day 29 comprises about 100 ng/mL GCSF, and at about day 29 onward GCSF is omitted.

In one embodiment of any one of the first to third aspects, the lymphocyte progenitor is a B-cell progenitor, and the method comprises culturing at the ALI for about 1, 2, 3, 4, 5 or 6 days, or about 1, 2, 3, 4, 5 or 6 weeks.

As used herein, a “WNT agonist” is a substance that mimics or increases WNT signaling. A WNT agonist is not to be restricted to a substance acting directly on WNT as the substance may act elsewhere in the WNT signaling pathway.

Non-limiting examples of WNT agonists include small molecules CHIR99021 (CAS 252917-06-9), a 2-amino-4,6-disubstituted pyrimidine, e.g. BML 284 (CAS 853220-52-7), SKL 2001 (CAS 909089-13-0), WAY 262611 (CAS 1123231-07-1), WAY 316606 (CAS 915759-45-4), SB 216763 (CAS 280744-09-4), IQ 1 (CAS 331001-62-8), QS 11 (CAS 944328-88-5), deoxycholic acid (CAS 83-44-3), BIO (CAS 667463-62-9), kenpaullone (CAS 142273-20-9), or a (hetero)arylpyrimidine. A WNT agonist may also be an agonist antibody or functional fragment thereof or an antibody-like polypeptide.

In one embodiment, the WNT agonist is CHIR99021 ((CHIR) CAS 252917-06-9), and the PSC is cultured in a medium comprising about 3 μM. In other embodiments, the medium may comprise about 2 μM, about 2.1 μM, about 2.2 μM, about 2.3 μM, about 2.4 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 4.1 μM, about 4.2 μM, about 4.3 μM, about 4.4 μM, about 4.5 μM, about 4.6 μM, about 4.7 μM, about 4.8 μM, about 4.9 μM, or about 5 μM CHIR.

H-1152 (EMD Millipore, catalog no. 555550) and Y27632 (Tocris, catalog no. 1254) are highly potent, cell-permeable, selective ROCK (Rho-associated coiled coil forming protein serine/threonine kinase) inhibitors. Rho-associated, coiled-coil containing protein kinase (ROCK) is involved mainly in regulating the shape and movement of cells by acting on the cytoskeleton. Thus, a “ROCK inhibitor” inhibits this function, and for present purposes a “ROCK inhibitor” enhances survival of iPSCs.

In one embodiment the ROCK inhibitor is Y27632 (CAS No: 129830-38-2). Other examples of ROCK inhibitors that may be used in the present method are AS 1892802 (CAS No: 928320-12-1), Fasudil hydrochloride (CAS No: 105628-07-7), GSK 269962 (CAS No: 850664-21-0), GSK 429286 (CAS No: 864082-47-3), H 1152 dihydrochloride (CAS No: 871543-07-6), Glycyl-H 1152 dihydrochloride (CAS No: 913844-45-8), HA 1100 hydrochloride (CAS No: 155558-32-0), OXA 06 dihydrochloride, RKI 1447 dihydrochloride, SB 772077B dihydrochloride (CAS No: 607373-46-6), SR 3677 dihydrochloride (CAS No: 1072959-67-1), and TC-S 7001 (CAS No: 867017-68-3).

The concentration of the ROCK inhibitor in which the cells are cultured may be about 0.1 μM, 0.5 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 40 μM, 50 μM, 60 μM, 70 μM, 80 μM, 90 μM, or 100 μM, for example.

As used herein, “NOTCH1 inhibitor” refers to an antagonist of NOTCH1 and includes substances acting directly or indirectly on NOTCH1, examples of which are known in the art. In one embodiment, the NOTCH1 inhibitor is a gamma secretase inhibitor. In one embodiment, the NOTCH1 inhibitor is N—[N-(3,5-difluorophenacetyl)-L-alanyl]-S-phenylglycine t-butyl ester (DAPT). Other examples of NOTCH1 inhibitors include N-[(1S)-2-[[(7S)-6,7-Dihydro-5-methyl-6-oxo-5H-dibenz[b,d]azepin-7-yl]amino]-1-methyl-2-oxoethyl]-3,5-difluorobenzeneacetamide (DBZ), Cyclohexyl 1,4,5,6,7,8-hexahydro-2,7,7-trimethyl-4-(4-nitrophenyl)-5-oxo-3-quinolinecarboxylate (FLI-06), benzeneacetamide (LY411575, CAS no. 209984-57-6), dibenzazepine (YO-01027, CAS no. 209984-56-5), propanediamide (R04929097, CAS no. 847925-91-1), Semagacestat (LY450139, CAS no. 425386-60-3), and antibodies that block NOTCH1 signalling, including antibodies directed to NOTCH1 (e.g. brontictuzumab) and NOTCH1 ligands (e.g. demcizumab).

Cells may be cultured in a medium comprising a NOTCH1 inhibitor, e.g. DAPT, concentration of about 1, 2, 3, 4, 5, 6, 7, 8, 9, 10, 11, 12, 13, 14, 15, 16, 17, 18, 19 or 20 μM.

As used herein, “CD117 activator” refers to a ligand capable of activating CD117, which is the receptor for SCF. Therefore, in one embodiment, a CD117 activator is SCF.

As used herein, “pluripotent stem cell” or “PSC” refers to a cell that has the ability to reproduce itself indefinitely, and to differentiate into any other cell type. There are two main types of pluripotent stem cell: embryonic stem cells (ESCs) and induced pluripotent stem cells (iPSCs).

As used herein, “embryonic stem cell” or “ESC” refers to a cell isolated from a five to seven day-old embryo donated with consent by subjects who have completed in vitro fertilization therapy, and have surplus embryos. The use of ESCs has been hindered to some extent by ethical concerns about the extraction of cells from human embryos.

Suitable ESCs for use in any of the aspects of the invention include, but are not limited to, H1 and H9 human ESCs.

As used herein, “induced pluripotent stem cell” or “iPSC” refers to an ESC-like cell derived from adult cells. iPSCs have very similar characteristics to ESCs, but avoid the ethical concerns associated with ESCs, since iPSCs are not derived from embryos. Instead, iPSCs are typically derived from fully differentiated adult cells that have been “reprogrammed” back into a pluripotent state.

Suitable human iPSCs for use in any of the aspects of the invention include, but are not limited to, iPSC 19-9-7T, MIRJT6i-mND1-4 and MIRJT7i-mND2-0 derived from fibroblasts and iPSC BM119-9 derived from bone marrow mononuclear cells. Other suitable iPSCs may be obtained from Cellular Dynamics International of Madison, Wis., USA.

As used herein, “PSC-derived CD34⁺ cell” refers to a cell produced from a PSC and that expresses cell surface marker CD34, i.e. is positive for CD34. A PSC-derived CD34⁺ cell can by purified or enriched according to methods known in the art, for example fluorescence-activated cell sorting (FACS) or anti-CD34 antibody columns.

As used herein, “embryoid body” and “EB” refers to a three-dimensional aggregate of PSCs. Advantageously, EBs in may cultured in suspension thus making EB cultures scalable for clinical applications. Additionally, the three-dimensional structure of EBs, including the establishment of complex cell adhesions and paracrine signaling within the EB microenvironment, enables differentiation and morphogenesis which yields microtissues that are similar to native tissue structures, for example the AGM as disclosed herein. An embryoid body may generate its own endogenous stromal cell.

In general, the invention relates to two multipotent cell types—PSCs and CD34⁺ cells. The relationship between the two cell types is that PSCs are the starting material used to make embryoid bodies, which then generate the CD34⁺ cells at around day 8.

In one embodiment, the PSCs of the present disclosure are cultured as an EB.

Methods for culturing EBs are known in the art. A specific example of a spin EB method that may be used in the present invention is described in Ng et al. Nature Protocols 3, 768-776 (2008). Alternatively, an example of a “swirler” EB method is disclosed herein.

As used herein, “medium” or its plural “media” refers to a liquid or gel designed to support the growth of cells. In some embodiments, the cell culture medium comprises APEL medium.

As used herein, “APEL” medium refers to the Albumin Polyvinylalcohol Essential Lipids medium described in Ng et al. Nature Protocols 3, 768-776 (2008). APEL medium is available commercially. In an embodiment of the first or fifth aspects of the invention, the medium is APEL medium.

All proteins described herein are known to the person skilled in the art, and most if not all proteins described herein are available commercially. Alternatively, the person skilled in the art is capable of producing the proteins recombinantly, or synthetically.

Although the presently disclosed media may include specific components (e.g. morphogens, small molecules, and hematopoietic cytokines), it is contemplated that other components with the same, equivalent, or similar properties may be used in addition to or in place of those disclosed, as are known in the art.

In some embodiments, the medium used to produce the PSC-derived CD34⁺ cell comprises 20-40 ng/mL BMP4, 20-30 ng/mL VEGF, 40 ng/mL SCF, 10-20 ng/mL ACTIVIN A, and 10 ng/mL FGF2.

In some embodiments, the concentration of BMP4 in the medium used to produce the PSC-derived CD34⁺ cell is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, or about 60 ng/mL.

In some embodiments, the concentration of VEGF in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, or about 70 ng/mL.

In some embodiments, the concentration of SCF in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 70 ng/mL, about 80 ng/mL, about 90 ng/mL, about 100 ng/mL, about 110 ng/mL, or about 120 ng/mL.

In some embodiments, the concentration of ACTIVIN A in the medium used to produce the PSC-derived CD34⁺ cell is about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, or about 30 ng/mL.

In one embodiment, GDF8 may be substituted for Activin A.

In some embodiments, the concentration of FGF2, also known as basic fibroblast growth factor (bFGF), in the medium used to produce the PSC-derived CD34⁺ cell is about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

In some embodiments, the concentration of IGF2 (IGFII) in the medium used to produce the PSC-derived CD34⁺ cell is about 20 ng/mL, about 25 ng/mL, about 30 ng/mL, about 35 ng/mL, about 40 ng/mL, about 45 ng/mL, or about 50 ng/mL. Preferably, the medium comprises IGF2 at 30 ng/mL.

In some embodiments, the concentration of IL-6 in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, or about 50 ng/mL. Preferably, the concentration of IL-6 in the medium is 25 ng/mL to 30 ng/mL.

In some embodiments, the concentration of IL-3 in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, or about 50 ng/mL. Preferably, the concentration of IL-3 in the medium is 25 ng/mL to 30 ng/mL.

In some embodiments, the concentration of TPO in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, or about 50 ng/mL. Preferably, the concentration of TPO in the medium is 25 ng/mL to 30 ng/mL.

In some embodiments, the concentration of FLT3L in the medium used to produce the PSC-derived CD34⁺ cell is about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, or about 70 ng/mL.

In some embodiments, the concentration of EPO in the medium used to produce the PSC-derived CD34⁺ cell is about 1 U/mL, about 2 U/mL, about 3 U/mL, about 4 U/mL, about 5 U/mL, about 6 U/mL, about 7 U/mL, about 8 U/mL, about 9 U/mL, or about 10 U/mL. Preferably, the medium comprises 3 U/mL, 4 U/mL or 5 U/mL EPO.

In some embodiments, the concentration of GM-CSF in the medium used to produce the PSC-derived CD34⁺ cell is about 10 ng/mL, about 15 ng/mL, about 20 ng/mL, about 25 ng/mL, about 30 ng/mL, about 35 ng/mL, about 40 ng/mL, about 45 ng/mL, or about 50 ng/mL. Preferably, the concentration of GM-CSF in the medium is 25 ng/mL.

Media disclosed herein may be also made in concentrated, including dried, forms that are diluted prior to use, such as 2×, 10×, 100×, or 1000× concentrations.

As used herein, “culturing” refers to the process by which cells are grown under controlled, in vitro conditions. It follows that “co-culturing” refers to the process by which two or more cell types are grown together under controlled, in vitro conditions.

As used herein, “extracellular matrix” refers to the non-cellular component of all tissues and organs that provides essential physical scaffolding for the cellular components and initiates crucial biochemical and biomechanical cues that are required for tissue morphogenesis, differentiation and homeostasis. It follows that an “extracellular matrix protein” is a protein present in and/or derived from the extracellular matrix. Examples of extracellular matrix proteins include collagens, elastin, vitronectin, fibronectin, laminin, and proteoglycans. Extracellular matrix proteins, including mixtures, are available commercially, e.g. MATRIGEL™.

Also disclosed is a method for treating a condition, disease or disorder comprising administering a cell of the disclosure. Also disclosed is use of a cell of the disclosure in the manufacture of a medicament for treating a condition, disease or disorder. Also disclosed is a cell of the disclosure for use in a method of treating a condition, disease or disorder.

It will be appreciated by the person skilled in the art that the exact manner of administering to a subject a therapeutically effective amount of a cell of or produced according to the invention for treating a condition, disease or disorder will be at the discretion of the medical practitioner. The mode of administration, including dosage, combination with other agents, timing and frequency of administration, and the like, may be affected by the diagnosis of a subject's likely responsiveness to treatment with the cell produced according to the invention, as well as the subject's condition and history.

As used herein, the term “therapeutic composition” refers to a composition comprising a cell produced according to the invention that has been formulated for administration to a subject. Preferably, the therapeutic composition is sterile. In one embodiment, the therapeutic composition is pyrogen-free.

The cell produced according to the invention will be formulated, dosed, and administered in a fashion consistent with good medical practice. Factors for consideration in this context include the particular type of condition, disease or disorder being treated, the particular subject being treated, the clinical condition of the subject, the site of administration, the method of administration, the scheduling of administration, possible side-effects and other factors known to medical practitioners. The therapeutically effective amount of the cell produced according to the invention to be administered will be governed by such considerations.

The cell produced according to the invention may be administered to a subject by any suitable method including intravenous (IV), intra-arterial, intramuscular, intraperitoneal, intracerobrospinal, subcutaneous (SC), intra-articular, intrasynovial, intrathecal, intracoronary, transendocardial, surgical implantation, topical and inhalation (e.g. intrapulmonary) routes. Most preferably, the cell produced according to the invention is administered IV.

The term “therapeutically effective amount” refers to an amount of the cell produced according to the invention effective to treat a condition, disease or disorder in a subject.

As used herein, “cell produced according to the invention” includes a lymphocyte progenitor cell and cells further differentiated therefrom, including, but not limited to, a T-cell with defined antigen specificity, optionally a CAR T-cell, or a B-cell.

The terms “treat”, “treating” or “treatment” refer to both therapeutic treatment and prophylactic or preventative measures, wherein the aim is to prevent or ameliorate a condition, disease or disorder in a subject or slow down (lessen) progression of a condition, disease or disorder in a subject. Subjects in need of treatment include those already with the condition, disease or disorder as well as those in which the condition, disease or disorder is to be prevented.

The terms “preventing”, “prevention”, “preventative” or “prophylactic” refers to keeping from occurring, or to hinder, defend from, or protect from the occurrence of a condition, a disease or disorder, including an abnormality or symptom. A subject in need of prevention may be prone to develop the condition, disease or disorder.

The term “ameliorate” or “amelioration” refers to a decrease, reduction or elimination of a condition, a disease or disorder, including an abnormality or symptom. A subject in need of treatment may already have the condition, disease or disorder, or may be prone to have the condition, disease or disorder, or may be in whom the condition, disease or disorder is to be prevented.

As used herein, the term “subject” refers to a mammal. The mammal may be a primate, particularly a human, or may be a domestic, zoo, or companion animal. Although it is particularly contemplated that the method and its resulting definitive progenitor cell or population of definitive progenitor cells disclosed herein are suitable for medical treatment of humans, they are also applicable to veterinary treatment, including treatment of domestic animals such as horses, cattle and sheep, companion animals such as dogs and cats, or zoo animals such as felids, canids, bovids and ungulates.

As used herein, “syngeneic” refers to biological material, e.g. cells or tissues, that are genetically sufficiently identical and immunologically compatible to allow for transplantation. It follows that as used herein, “non-syngeneic” biological material, e.g. cells or tissues, that are not genetically sufficiently identical and immunologically compatible to allow for transplantation.

As used herein, “stromal cell” refers to a cell that supports the function of another cell type in co-culture. Stromal cells may be connective tissue cells, for example uterine mucosa (endometrium), prostate, bone marrow, lymph node or ovary. Examples of stromal cells include fibroblasts and pericytes. Without wishing to be bound to any particular hypothesis, stromal cells have been described to be involved in human haematopoiesis and inflammatory processes by regulating local cytokine networks.

Examples of stromal cells include an OP9, MS5 or S17 cell. In one embodiment, the stromal cell expresses delta like canonical notch ligand 4 (DLL4). In one embodiment, the stromal cell is an OP9-DLL4 cell.

In one embodiment of the method of the first aspect, culturing the CD34⁺ cell excludes co-culturing the CD34⁺ cell with an “exogenous”, or added, stromal cell. Without wishing to be bound to theory, it is thought that the cultures generate their own stromal cells, thereby avoiding the need to add stromal cells, which are usually mouse derived, for support. That is, use of exogenous stromal cells is avoided.

### EXAMPLES

### Example 1

A. Human Pluripotent Stem Cell Culture

In general, basic culture of undifferentiated PSCs was performed as described (Costa et al., 2007; Kao et al., 2016). A brief summary is provided below.

ESCs and iPSCs were routinely grown in PSC medium consisting of: DMEM-F12 (Invitrogen); 20% knock-out serum replacement; 1× non-essential amino acids; 1× L-glutamine or L-alanyl-L-glutamine (e.g. GLUTAMAX); 0.1 mM β-mercaptoethanol and bFGF (10 ng/mL for ESCs, 50 ng/mL for iPSCs) in the presence of mouse embryonic fibroblasts (MEFs)(Costa et al., 2007).

On reaching ˜80% confluency, ESCs and iPSCs were passaged using EDTA dissociation buffer or TRYPLESELECT (Gibco). Briefly, medium was aspirated from the flask of PSCs and rinsed with PBS.

EDTA dissociation buffer or TRYPLESELECT was added and the flask was placed in the 37° C. incubator for 3 min.

The cells were then transferred to a centrifuge tube in PSC media and centrifuged for 3 min at 1500 rpm.

The medium was aspirated and the pellet was resuspended into fresh PSC medium.

The cells were transferred to new flasks at ˜15 000 cells/cm². The flasks had been previously seeded with fresh MEFs.

Alternatively, after incubation with the dissociation enzyme (EDTA or TRYPLESELECT), while the cells were not completely detached from the flask, the enzyme was aspirated first. Then, the cells were disassociated directly in PSC medium, and subsequently transferred to previously prepared flasks.

B. Hematopoietic Differentiation of PSCs

B.1—Phase 1 (EB Formation & Differentiation)

Day −1

The day prior to initiation of differentiation, PSCs were passaged onto new flasks containing fresh feeders (see section A).

Spin EBs (Day 0-8)

Day 0

Round bottom non-adherent 96-well plates were prepared by filling the outermost wells with 80 μl of distilled water in order to maintain humidity in the plate and prevent the medium from evaporating during the course of the differentiation experiment.

STAGE I differentiation medium was prepared: APEL+Activin A (20 ng/mL)+BMP4 (20 ng/mL)+VEGF (25 ng/mL)+SCF (25 ng/mL) (refer to (Ng et al., 2008) for preparation of APEL medium).

Following treatment with ACUTASE® to generate a single cell suspension, PSCs were collected in PBS, pelleted by centrifugation and resuspended into STAGE I medium to give a final concentration of 5000 cells/80 μl. 80 μl of the cell suspension was then aliquoted into each well of the 96 well plate, other than the outermost wells, using a multi-channel pipette.

The plates were centrifuged at 5000 rpm for 3 minutes to promote aggregation of the PSCs at the bottom of each well. The 96 well plates containing the forming spin EBs were then placed in a 37° C. incubator with 5% CO₂.

Day 4

Leaving the EBs in the wells, STAGE I medium was carefully removed by aspiration. The medium was changed to STAGE II APEL medium, containing VEGF (50 ng/mL), SCF (100 ng/mL), IGFII (30 ng/mL), and FGF2 (10 ng/mL).

Day 7

Using a multi-channel pipette, the EBs were removed from the 96 well plates and transferred to 50 ml centrifuge tube. EBs were allowed to settle. The medium was removed and replaced with STAGE III APEL medium, containing Flt3L (50 ng/mL), SCF (50 ng/mL) and IL7 (20 ng/mL), VEGF (50 ng/mL) and bFGF (20 ng/mL). The EBs were then transferred to pre-gelatinized 6 well tissue culture treated plates (30 EBs/well) and subsequently returned to a tissue culture incubator (37° C., 5% CO₂)

Day 12 Onwards

Every 3-4 days, half of the medium was changed with fresh STAGE III medium.

Swirler EBs (Day 0-8˜15)

Swirler medium (I) comprised 0.1% (or 0.25%) ALBUCULT® (Novozymes), 0.1% methylcellulose (MC), 0.1% polyvinyl alcohol (PVA), GLUTAMAX, ascorbic acid-2-phosphate (AA2P, Insulin-Transferrin-Selenium-Ethanolamine (ITS-X, Gibco)), linoleic acid and linolenic acid (each 100 ng/mL), synthetic cholesterol (2.2 μg/mL), 2-mercaptoethanol 22 nM (or 55 nM), and protein free hybridoma medium II (PFHM II) (4%) in IMDM/F12 media.

Day 0

Stage I medium was prepared by adding Rock Inhibitor (final concentration of 10 μm, 20 μm for H9 cell line), CHIR (0.5 μm), Activin A (10 ng/mL), BMP4 (40 ng/mL), SCF (20 ng/mL), VEGF (20 ng/mL) and bFGF (5 ng/mL) into swirler media (I).

PSC culture medium was aspirated from the cell culture flask and the adherent cells rinsed with PBS.

Cells were treated with ACUTASE® or EDTA dissociation buffer for 3 minutes at 37° C.

Without agitation, ACUTASE® was aspirated from the flask and the cells were harvested by tapping the flasks. Stage I medium was directly added to the dislodged cells. Following a brief period of pipetting to remove all the cells and to break up large cell clumps, the mixture was immediately transferred to non-tissue culture treated 6 cm dishes (5 ml per dish). (When EDTA dissociation buffers was used, the cells were passed through the cell strainer cap (pore size: 40 μm) prior to transferring to the 6 cm dishes.)

The 6 cm dishes were then placed on a rotating platform located inside a 37° C. incubator. Swirler cultures were set to swirl at 60 rpm.

Day 1

Formation of small EBs was confirmed by viewing the cultures under a standard tissue culture microscope.

From this stage onwards, Swirler medium (II) can be used instead of Swirler media (I). The Swirler medium (II) can be prepared by omission of PVA & MC from Swirler medium (I), and optional addition of Pluronic F-68 (1×). Stage II medium contained CHIR (0.5 μm), bFGF (10 ng/mL) BMP4 (40 ng/mL), SCF (20 ng/mL) and VEGF (20 ng/mL).

Medium containing the EBs was transferred to a tissue culture tube and the EBs allowed to settle at the bottom of the tube for 5 to 10 minutes (if necessary, the tube was centrifuged at 700 rpm for 2 min to pellet the EBs).

Medium was carefully aspirated from the tube and replaced with Stage II medium.

The mixture was then transferred into 6 cm dishes and set to swirl.

Day 2-4

SB-431542 (3 μm) and CHIR-99021 (3 μm) was added to Stage II medium, and the medium changed as described above.

Medium was changed daily.

Day 4˜8

Mesoderm differentiation was confirmed by flow cytometry analysis using anti-CD13 & anti-EPCAM antibodies.

Stage III medium was used between day 4˜8. Stage III medium: Swirler media (I) or (II)+BMP4 (20 ng/mL), bFGF (10 ng/mL), VEGF (50 ng/mL), SCF (50 ng/mL) and IGFII (20 ng/mL).

Medium was changed every second day.

Day 8˜15

After confirming the generation of hemato-endothelial progenitors via flow cytometry analysis using anti-CD34 antibody, the medium was changed to Stage IV medium composed of: Swirler medium (I) or (II) plus BMP4 (20 ng/mL), bFGF (10 ng/mL), VEGF (25 ng/mL), SCF (100 ng/mL), IGFI (20 ng/mL), IL3 (50 ng/mL), FLT3L (25 ng/mL) and TPO (25 ng/mL).

Media was Changed Every Three Days.

B.2—Phase 2 Hematopoietic Differentiation

Phase 2 medium was prepared as required for each experiment.

Liquid Culture System

Cell culture plates were coated with a 10 μg/mL solution of vitronectin (1 μg/cm²) in PBS for at least 2 hours prior to use.

EBs were collected into a tissue culture tube and allowed to settle as described above.

After the EBs were settled at the bottom of the tube, the medium was aspirated.

EBs were resuspended in phase 2 medium and transferred to vitronectin coated plates.

Differentiation medium was changed every 4˜5 days.

Air-Liquid Interface (ALI) Culture System

Corning Transwells:

The air liquid interface (ALI) membranes were coated with a 10 μg/mL solution of vitronectin (1 μg/cm²) in PBS for at least 2 hours prior to use.

Prior to transfer of the EBs, Phase 2 medium was added to wells (1 ml/well for 6 well plates and 500 μl/well for 12 well plates).

EBs were resuspended in APEL medium and transferred onto ALI membranes in a minimal volume of APEL medium (˜200 μl/well for 6-well plates and ˜100 μl/well for 12-well plates).

The next day, EBs could be seen to have attached to the membranes, and the excess medium used to transfer the EBs was aspirated.

Medium within the well below the membrane was changed every 4 days.

Merck Membranes

Isopore membranes (RTTP01300) were purchased from Merck PL.

To coat the membranes with vitronectin, a 10 cm dish was covered with vitronectin containing water (10 μg/mL). The membranes were then transferred to the 10 cm dish and allowed to float for 1 day.

To set up the ALI culture configuration, the well of an organ culture was filled with 1 ml of phase 2 medium and the vitronectin coated membrane was suspended over the medium (the side of membrane which was coated with vitronectin was facing up). The EBs were resuspended in 50˜100 μl APEL medium and transferred onto these ALI membranes.

The medium was changed every 4˜5 days. To change the medium, the membranes were carefully folded from an edge using sterile tweezers. While keeping the membrane folded with one hand, the medium was carefully aspirated from the well and the fresh medium then added.

T-Cell Differentiation on Air Liquid Interface (ALI)

From day 8 of differentiation, when the cells were first transferred onto ALI, fresh medium was added every three days. The composition of medium changed at day 8, 16 and 24 as follows

Day 8˜

The medium was removed and replaced with APEL medium/Swirler medium (I) or (II), containing VEGF (50 ng/mL), SCF (100 ng/mL), bFGF (10 ng/mL), FLT3L (10 ng/mL), IL3 (10 ng/mL) and IL7 (20 ng/mL).

Day 16˜

Medium was changed to APEL medium/Swirler medium (I) or (II), including VEGF (50 ng/mL), SCF (20 ng/mL) and IL7 (20 ng/mL).

Day 24˜

Medium was changed to APEL medium/Swirler medium (I) or (II), including VEGF (50 ng/mL) and IL7 (20 ng/mL).

Co-Culture Systems Involving DLL4 Stromal or OP9 Stromal Cells for Production of B-Cell Progenitors

Co-culture systems generally used alpha-MEM medium supplemented with 10˜20% fetal calf serum (FCS), Penicillin Streptomycin, L-glutamine and 2-Mercaptoethanol (0.1 mM).

Stromal cells were seeded into an appropriate number of wells of a multi-well plate and allowed to grow until they reached confluency.

Day 8˜15

The medium was changed to Swirler medium (I) or (II); supplemented with: BMP4 (20 ng/mL), bFGF (10 ng/mL), VEGF (25 ng/mL), SCF (100 ng/mL), IGFI (20 ng/mL), IL3 (50 ng/mL), FLT3L (25 ng/mL) and TPO (25 ng/mL). Medium was changed every three days.

Day 12-18˜

At around day 15, cells were transferred onto confluent layers of OP9 or OP9-DLL4 stromal cells and cultured in the following conditions:

Medium: alpha-MEM medium supplemented with 5˜10% FCS, penicillin streptomycin, L-glutamine and 2-mercaptoethanol (0.1 mM)

Cytokines: SCF (50 ng/mL) and DAPT (10 uM). (When OP9 cells were used, addition of DAPT is optional. However, the presence of DAPT enhances the development of B-lymphocytes.)

Media was changed every 4˜5 days.

Every 7˜10 days, the culture was passaged onto new wells coated with fresh layers of stromal cells.

To passage the cells, the cultures were detached from the wells by pipetting up and down (no enzymatic digestion was required). The cell suspension was then filtered through cell 35 μm strainer and centrifuged for 3 min at 1500 rpm.

The old medium was aspirated and the pellet resuspended in fresh medium.

B-Cell Differentiation on ALI

At day 8 of differentiation, the EBs were transferred onto vitronectin coated plates and grown in APEL, Swirler medium (I) or (II) and in the presence of the following cytokines:

VEGF (50 ng/mL), SCF (100 ng/mL), bFGF (10 ng/mL), FLT3L (10 ng/mL) and IL3 (10 ng/mL).

Medium was changed every three days.

Day 15-20˜

Once the generation of hematopoietic cells was observed (visual observation under the microscope) at around day 15 to day 20 of differentiation, the cytokines were changed as follows (the medium remains the same):

VEGF (50 ng/mL), SCF (100 ng/mL), bFGF (10 ng/mL) and DAPT (10 μM). In addition, IL3 (10 ng/mL), IL6 (10 ng/mL), G-CSF (10 ng/mL), and IL7 (10 ng/mL) were sometimes included.

Medium was changed every three days.

C. Flow Cytometry Analysis

FACS wash=PBS+5% FCS

PI media=FACS wash+Propidium Iodide (1 μg/mL)

Following TRYPLESELECT treatment (minimum 10 min for day 8 EBs and later cultures), cells were harvested in FACS wash and passed through the cell strainer caps of a FACS tube to obtain the single cell suspension. In cases where cells were harvested from co-culture conditions, cells were harvested by physical agitation (pipetting up and down) as described for cell passaging (Section A).

The FACS tubes were centrifuged for 3 min at 1500 rpm.

The medium was removed and FACS wash containing appropriate combinations of antibodies was added and the samples incubated on ice for 15 min.

The cells were then washed twice with 4 ml of FACS wash (with centrifugation between each wash).

FACS wash containing 1 μg/mL PI (0.2-0.4 ml per sample) was added to FACS tubes prior to analysis.

Where un-conjugated antibodies were used, single cells were first stained with primary antibodies for up to 30 min on ice. Cells were washed twice with FACS wash and then the secondary antibodies added. Following incubation for a further 30 min and twice rinsing the cells with FACS wash, additional conjugated antibodies were used as required.

After a final centrifugation step, the cells were resuspended in PBS prior to flow cytometry analysis. Note that no PI was added when performing intacellular FACS

D. Real-Time PCR (qPCR)

RNA was extracted from purified populations using Isolate II RNA micro kit and the method suggested by manufacturer (catalog no. BIO-52075, Bioline). cDNA was prepared using the Tetro cDNA synthesis kit (catalog no. BIO-65042, Bioline) according to instructions provided with the kit.

qPCR analysis was conducted on ABI Real-time PCR (Applied Biosystems) machine. In a MicroAmp 96-well optical reaction plate, 20 μl reaction samples were prepared containing cDNA, Taqman universal PCR master mix and Taqman assay probes. The plate was sealed with optical adhesive and transferred to qPCR machine.

Data was analysed by ABI 7300 software.

E. Immunofluorescence Analysis

In preparation for immunofluorescence analysis, cells were plated into 24 well plates.

The next day, culture medium was aspirated and the cells were rinsed with PBS.

Fixation and permeabilization of the cells was performed simultaneously. A PBS solution containing paraformaldehyde (final concentration: 4%) and triton-X (final concentration: 0.5%) was prepared and added to the cells.

After 10 min incubation at room temperature, the solution was aspirated and the cells were washed with PBS 3 times.

During the incubation, blocking buffer was prepared. Human/goat/donkey serum (Ideally the source of serum should match the species of the host in which the secondary antibodies were raised) were mixed in Perm/Wash buffer (1K) (provided within BD kit, Catalog no. 554714), giving the final concentration of 100 μg/mL of serum in solution.

Following another rinse with cold PBS, blocking buffer was added and the sample incubated for 10 min at room temperature.

During the blocking step, the primary antibody mixture was prepared in Perm/Wash buffer (1×)

Blocking buffer was aspirated and replaced with the primary antibody mixture which constituted Perm/wash solution and antibodies at dilutions indicated in Section J.

After 1˜2 hours incubation at room temperature, the antibody mixture was removed and the cells were rinsed with Perm/Wash three times.

Secondary antibodies and DAPI (final concentrations were 1:1000 and 3 μM, respectively) were then added to the Perm/Wash solution and this was then added to the cells. The plates were placed in the dark and incubated for 1 hour at room temperature.

The cells were washed with Perm/Wash 3 times and PBS was added to wells. (If a conjugated antibody was used in combination with un-conjugated antibodies, staining with the conjugated antibody was started at this stage. In this case, samples were taken through the same series of steps described above.)

F. Agarose Gel Electrophoresis

DNA samples were separated by electrophoresis on 1% agarose gel that contained 1×SAFERED DNA dye (catalog no. 533102, ThermoFisher Scientific). The size of PCR products was determined relative to DNA molecular weight markers (1 Kb ladder—ThermoFisher Scientific MASSRULER) by placing the gel on a UV light box and capturing the gel image using GENE GENIUS Bio Imaging System.

G DNA Extraction from Human Cell Lines

For DNA extraction, cell lysis buffer was prepared first. Cell lysis buffer included: Water+Tris HCL pH 8.5 (10 mM), EDTA (5 mM), SDS (0.2%), NaCl (0.2 mM). Prior to use, Proteinase K (0.1 mg/mL) was added to the lysis buffer.

Cells were treated with lysis buffer at 37° C. for one night.

The next day, the lysed cell mixture was transferred to a 1.5 ml microfuge tube. Two volumes of phenol-chloroform 1:1 solution was added to each sample and the sample mixed by vortexing. The sample was centrifuged for 2.5 min at 14 000 rpm to separate the aqueous and organic layers. The upper aqueous phase was collected carefully and transferred into new microfuge tube containing 2.5 volumes of 100% ethanol. 0.1 volume of 3 M sodium acetate (0.3 M final) was added to the sample. The solution was mixed and centrifuged for 15 min (maximum speed) to collect the precipitated DNA. The supernatant was removed from the tube and the DNA pellet was washed with 200 μl of 70% ethanol. The sample was centrifuged for 5 mins (maximum speed) and the 70% ethanol was discarded.

The dried DNA sample was dissolved in 50 μl TE/EB buffer.

H. Imaging

A ZEISS fluorescence microscope and LSM 780 confocal microscope was used for imaging. The images were analysed and prepared using Zen software provided by ZEISS.

I. Concentration of Cytokines

The cytokines were added as described above. The tables below indicate the concentration of cytokines used during different experiments.

All concentrations are in ng/mL unless otherwise mentioned.

T-Cell Differentiation

TABLE 1
Spin EB (Day 0-8), ALI & Vitronectin
coated plates (Day 8 onward):

| | Day | Day | Day | Day | Day | Day 22 |
|---|---|---|---|---|---|---|
| Cytokines | 0-2 | 2-4 | 4-8 | 8-15 | 15-22 | onward |
| Activin A | 10 | 10 | — | — | — | — |
| BMP4 | 20 | 20 | 20 | 10 | 10 | 10 |
| VEGF | 25 | 25 | 50 | 50 | 50 | 25 |
| SCF | 25 | 25 | 50 | 100 | 50 | 25 |
| bFGF | 10 | 10 | 10 | 10 | 10 | 10 |
| SB | — | 3 μM | — | — | — | — |
| CHIR | 0.5 μM | 3 μM | — | — | — | — |
| IGF II | — | — | 20 | — | — | — |
| Flt3l | — | — | — | 10 | 10 | 10 |
| IL7 | — | — | — | 20 | 20 | 10 |
| IL6 | — | — | — | 20 | 20 | 10 |
| IL3 | — | — | — | 20 | 20 | 10 |
| IL2 | — | — | — | 20 U/mL | 20 U/mL | 20 U/mL |

TABLE 2
Swirler EBs (Day 2 onward is as described in Table 1)

| Cytokines | Day 0-1 | Day 1-2 |
|---|---|---|
| Activin A | 10 | — |
| BMP4 | 40 | 20 |
| VEGF | 20 | 20 |
| SCF | 20 | 20 |
| bFGF | 5 | 10 |
| SB | — | — |
| CHIR | 0.5 μM | 0.5 μM |
| Y27632 | H9: 20 μM | |
| | iPS: 10 μM | — |

TABLE 3
T-cell differentiation from CD34+
HPCs on OP9-DLL4 stromal cells

| Cytokines | Week 1 | Week 2 | Week 3 | Week 4 onward |
|---|---|---|---|---|
| SCF | 100 | 50 | 20 | 10 |
| Flt3l | 10 | 10 | 10 | 10 |
| IL7 | 10 | 10 | 10 | 10 |

B-Cell Differentiation

TABLE 4
Spin EBs:

| | Day | Day | Day | Day | Day | Day | Day 29 |
|---|---|---|---|---|---|---|---|
| Cytokines | 0-2 | 2-4 | 4-8 | 8-15 | 15-22 | 22-29 | onward |
| Activin A | 10 | 10 | — | — | — | — | — |
| BMP4 | 20 | 20 | 20 | 20 | — | — | — |
| VEGF | 20 | 20 | 50 | 25 | — | — | — |
| SCF | 20 | 20 | 50 | 100 | 100 | 50 | 20 |
| bFGF | 10 | 10 | 10 | 10 | — | — | — |
| SB | — | 3 μM | — | — | — | — | — |
| CHIR | 0.5 μM | 3 μM | — | — | — | — | — |
| IGF II | — | — | 20 | — | — | — | — |
| DAPT | — | — | — | — | 10 μM | 10 μM | 10 μM |
| IGF I | — | — | — | 20 | — | — | — |
| Flt3l | — | — | — | 25 | 20 | 10 | 10 |
| IL7 | — | — | — | — | 10 | 10 | 5 |
| TPO | — | — | — | 25 | 20 | 10 | — |
| IL3 | — | — | — | 50 | 10 | 10 | — |
| SDF I | — | — | — | — | 100 | 100 | — |
| G-CSF | — | — | — | — | 10 | 10 | 10 |

TABLE 5
Swirler EBs (Day 2 onward is as described in Table 4)

| Cytokines | Day 0-1 | Day 1-2 |
|---|---|---|
| Activin A | 10 | — |
| BMP 4 | 40 | 20 |
| VEGF | 20 | 20 |
| SCF | 20 | 20 |
| bFGF | 5 | 10 |
| SB | — | — |
| CHIR | 0.5 μM | 0.5 μM |
| Y27632 | H9 ESCs: 20 μM | |
| | iPSCs: 10 μM | — |

J. Cytokines & Antibodies

BMP4 and Activin A were purchased from R&D Systems, all other cytokines were purchased from PEPROTECH.

Antibodies were purchased from BD Biosciences and BioLegend.

TABLE 6
Antibodies used in the examples

| Antibody | Clone | Dilution |
|---|---|---|
| APC anti-CD7 | M-T701 | 1:40 |
| PE/PeCy7 anti-CD235a | HI 264 | 1:2000 |
| APC/PeCy7 anti-CD19 | H1B19 | 1:20 |
| APC anti-CD31 | WM59 | 1:100 |
| APC anti-CD4 | RPA-T4 | 1:40 |
| Bv/PeCy7 anti-Epcam | 9C4 | 1:50 |
| APC anti-CD43 | CD43-10G7 | 1:50 |
| APC/Bv anti-CD3 | UCHT1 | 1:20 |
| PeCy7 anti-CD5 | UCHT2 | 1:40 |
| PeCy7 anti-CD8 | SK1 | 1:40 |
| PE/PeCy7 anti-CD34 | 581 | 1:100 |
| PE/Bv anti-CD45 | HI30 | 1:20 |
| Bv/PE anti-CD10 | HI10a | 1:20 |
| Bv anti-CD45RA | HI100 | 1:20 |
| FITC anti-CD9 | M-L13 | 1:20 |
| PE anti-TCR α/β | IP26 | 1:20 |
| APC/PE anti-CD13 | WM15 | 1:50 |
| Bv anti-CXCR4 | 12G5 | 1:30 |
| APC/PE/Bv anti-Ckit | 104D2 | 1:20 |
| Bv anti-IL7Ra | A019D5 | 1:20 |
| APC anti-PAX5 | 1H9 | 1:100 |
| APC anti-Flag | L5 | 1:100 |
| anti-ERa | MC-201 | 1:100 |

### Example 2

In order to examine the authenticity of in vitro derived T-cells, the ability of T-cell progenitors generated according to Example 1, were tested for homing to the thymus of immunocompromised mice. A key property of T-cell precursors generated in either in vivo or in vitro is their capacity to home to, and undergo further development, in the thymus.

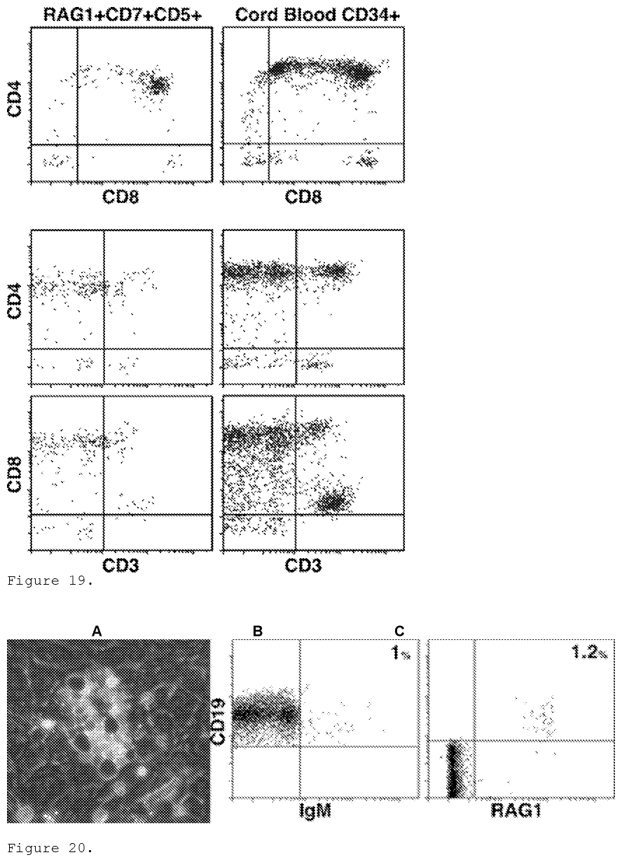

In order to test this function, cell fractions representing specific stages of early T-cell development, including RAG1⁺CD5⁺CD7⁺, RAG1⁻CD5⁺CD7⁺ were transplanted into non-irradiated immunocompromised mice. Only the RAG1⁺CD5⁺CD7⁺ cells homed to thymus. Human cells within the thymus of these mice, identified by human CD45, expressed combinations of CD3, CD4 and CD8 in proportions that mirrored that seen in the thymus of mice transplanted with unfractionated cord blood (FIG. 19). Interestingly, thymocytes derived from this in vitro system no longer expressed RAG1 (GFP), suggesting that TCR recombination had ceased.

### Example 3

As an alternative to using OP9 stromal cells expressing DLL4 (DLL4-0P9) for B-cell progenitor production according to Example 1, which requires addition of a NOTCH1 inhibitor such as DAPT to the culture medium to permit B-cell development, OP9 stromal cells (without DLL4) can also be used (FIG. 20). This system generates a larger number of B-cell progenitors (FIG. 20B), a small fraction of which (˜1%) are IgM⁺ (FIG. 20B). This frequency of IgM⁺ cells is similar to that seen with cord blood CD34⁺ cells cultured in similar systems. This system can be further enhanced by addition of DAPT, indicating residual NOTCH signalling from OP9 cells can restrict B-cell development.

### Example 4

As a further alternative to using stromal cells for B-cell progenitor production according to Example 1, small numbers of RAG⁺CD9⁺CD10⁺ B-cell progenitors can be generated in stromal cell-free ALI cultures supplemented with DAPT (anti-Notch) from around day 15 (FIG. 20C). Interestingly, these B-cell progenitors have only been observed outside vascular structures.

1. A method for producing a lymphocyte progenitor, the method comprising culturing a pluripotent stem cell (PSC)-derived CD34⁺ cell at an air-liquid interface (ALI).

2. The method of claim 1, wherein culturing the CD34⁺ cell excludes co-culturing the CD34⁺ cells with an exogenous stromal cell.

3. The method of claim 1, wherein the method excludes purifying the CD34⁺ cell before culturing.

4. The method of claim 1, comprising generating an embryoid body.

5. The method of claim 1, comprising culturing at the ALI for about 2 weeks to about 5 weeks.

6. The method of claim 1, wherein the PSC is human, optionally an embryonic stem cell (ESC) or induced PSC (iPSC).

7. The method of claim 1, wherein the lymphocyte progenitor is a T-cell progenitor, and wherein the method comprises culturing the CD34⁺ cell at the ALI in a medium comprising vascular endothelial growth factor (VEGF), basic fibroblast growth factor (bFGF, FGF2), stem cell factor (SCF), fms-related tyrosine kinase 3 ligand (FLT3L), and interleukin-7 (IL-7), and optionally IL-3.

8. The method of claim 7, wherein the medium excludes IL-3 or IL-6.

9. The method of claim 7, wherein an embryoid body is generated by culturing the PSC cell in a medium comprising a WNT agonist, optionally CHIR99021, and BMP4 and Activin A.

10. The method of claim 9, wherein the embryoid body is generated by culturing the PSC in a medium comprising a WNT agonist, BMP4 and Activin A from about day zero to about day 2, or from about day 2 to about day 4, or from about day zero to about day 4.

11. The method of claim 1, wherein the lymphocyte progenitor is a B-cell progenitor, and comprising culturing the CD34⁺ cell in a medium comprising a NOTCH1 inhibitor during or after an embryoid body comprising the CD34⁺ cell is cultured at the ALI.

12. The method of claim 11, comprising culturing the embryoid body comprising the CD34⁺ cell in a medium comprising the NOTCH1 inhibitor from about day 15, optionally to about day 32, during culturing at the ALI.

13. The method of claim 11, comprising culturing the embryoid body comprising the CD34⁺ cell at the ALI in a medium comprising a CD117 activator.

14. The method of claim 13, comprising culturing the embryoid body comprising the CD34⁺ cell in the medium comprising a CD117 activator.

15. The method of claim 13, further comprising culturing in a medium comprising IL-7 from about day 8.

16. The method of claim 15, comprising culturing in a medium comprising IL-7 for up to 35 days, optionally 30 days.

17. The method of claim 11, wherein the NOTCH1 inhibitor is N—[N-(3,5-difluorophenacetyl)-L-alanyl]-S-phenylglycine t-butyl ester (DAPT).

18. The method of claim 13, wherein the CD117 activator is stem cell factor (SCF).

19. A lymphocyte progenitor when produced by the method of claim 1.

20. Use of a lymphocyte progenitor when produced by the method of claim 1 in the manufacture of (i) a T cell with defined antigen specificity, optionally a chimeric antigen receptor (CAR) T cell, or (ii) an antibody.

21.-39. (canceled)
